(19) United States
(12) Patent Application Publication
(10) Pub. No.: US 2026/0191861 A1
(43) Pub. Date: Jul. 9, 2026

(54) INFLAMMASOME AND VEGF INHIBITION FOR RETINAL VASCULAR DISEASES

(71) Applicants: University of Virginia Patent Foundation, Charlottesville, VA (US); Universidade Federal de São Paulo, São Paulo (BR)

(72) Inventors: Jayakrishna Ambati, Charlottesville, VA (US); Felipe Pereira, Balneario Camboriu (BR); Michel Eid Farah, São Paula (BR); Eduardo Rodrigues, Florianópolis (BR)

(21) Appl. No.: 19/136,580

(22) PCT Filed: Dec. 7, 2023
(86) PCT No.: PCT/US2023/082862

§ 371 Date: Jun. 6, 2025

Related U.S. Application Data
(60) Provisional application No. 63/386,358, filed on Dec. 7, 2022.

(51) Int. Cl. A61K 31/513; A61K 31/7072; A61K 39/00; A61K 45/06; A61P 27/02; C07K 16/22

(52) U.S. Cl. CPC A61K 31/513; A61K 31/7072; A61K 45/06; A61P 27/02; C07K 16/22; A61K 2039/505

(57) ABSTRACT

The Diabetic Retinopathy Clinical Research Network (DRCR.net) Protocol T study demonstrated that monthly intravitreous injections of anti-vascular endothelial growth factor (VEGF) agents are effective in treating diabetic macular edema (DME) involving the center of the macula (center-involved DME (CI-DME)). These expensive and often burdensome injections also carry risks of vision-threatening complications. Provided herein it is demonstrated that orally administered lamivudine, an inflammasome inhibitor, improved vision.

### PRIORITY

This application claims priority to and the benefit of U.S. Provisional Patent Application Nos. 63/386,358, filed Dec. 7, 2022, the entire disclosure of which is incorporated herein by this reference.

### GOVERNMENT FUNDING

This invention was made with government support under R01EY031039 awarded by the National Institutes of Health. The government has certain rights in the invention.

### BACKGROUND

Retinal vascular diseases such as diabetic macular edema, wet age-related macular degeneration, branch retinal vein occlusion, and central retinal vein occlusion result in vision loss due to a combination of retinal edema (swelling) and disruption of retinal function.

### SUMMARY

Vascular endothelial growth factor (VEGF) inhibitors reduce retinal edema and improve vision, but often unsatisfactorily. Inflammasome inhibitors improve visual acuity without reducing retinal edema. Inflammasome inhibitors in combination with VEGF inhibitors improve visual acuity better than VEGF inhibition alone. Combination therapy or lower dose inflammasome inhibition, with or without VEGF inhibitors can treat retinal vascular diseases.

One aspect provides a method to prevent or treat a retinal vascular disease comprising administering one or more inflammasome inhibitors to a subject in need thereof. One aspect further comprises administering one or more vascular endothelial growth factor (VEGF) inhibitors. In one aspect, the one or more inflammasome inhibitors comprises a nucleotide reverse transcriptase inhibitors (NRTI) or an NRTI derivative. In one aspect, the NRTI derivative comprises an alkylated NRTI. In one aspect, the NRTI or alkylated NRTI comprises kamuvudine-9 (K-9), lamivudine (3TC), azidothymidine (AZT), kamuvudine-8 (K-8; 2-ethyl-AZT) or a combination thereof. In another aspect, the administered dose of the NRTI or NRTI derivative is equal to or less than 150 mg once or twice daily. In one aspect, the one or more VEGF inhibitors comprises aflibercept, bevacizumab, ranibizumab, conbercept, ziv-aflibercept, Razumab, Bvooviz, Cimerli, Xlucane, R-TPR-024, SIP-0133, UBTO10, CKD-701, SB15, MYL1701, ABP-938, FVB203, SOK583419, CT-P42, ALT-L9, OT-702, or Bevacizumab-vikg. In one aspect, the one or more inflammasome inhibitors and/or the one or more VEGF inhibitors are administered orally or injected. In one aspect, the one or more inflammasome inhibitors and/or the one or more VEGF inhibitors are injected in the eye of the subject.

In one aspect, the one or more inflammasome inhibitors comprises a compound of structural Formula (I)

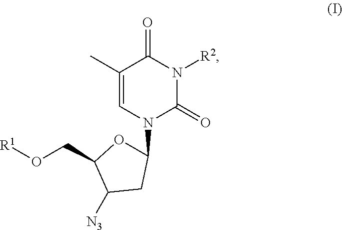

wherein:
R¹ is C₁₋₄ alkyl; and
R² is H or C₁₋₄ alkyl, provided that when R² is H, R¹ is not CH₃
or a salt thereof.

In one aspect, R² is CH₃ or CH₂CH₃. In one aspect, R¹ is n-C₄H₉.

In one aspect, the one or more inflammasome inhibitors comprises one or more of

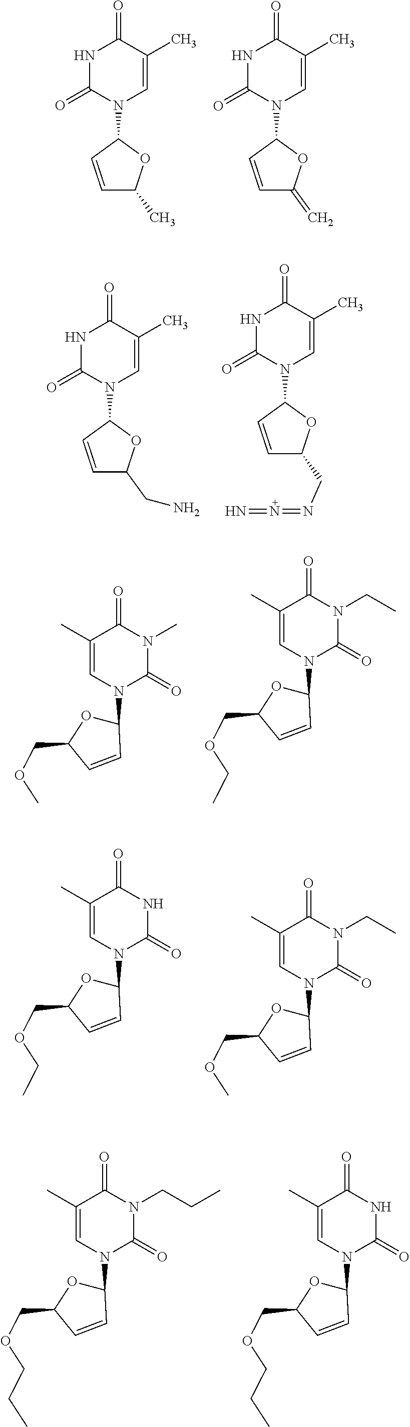

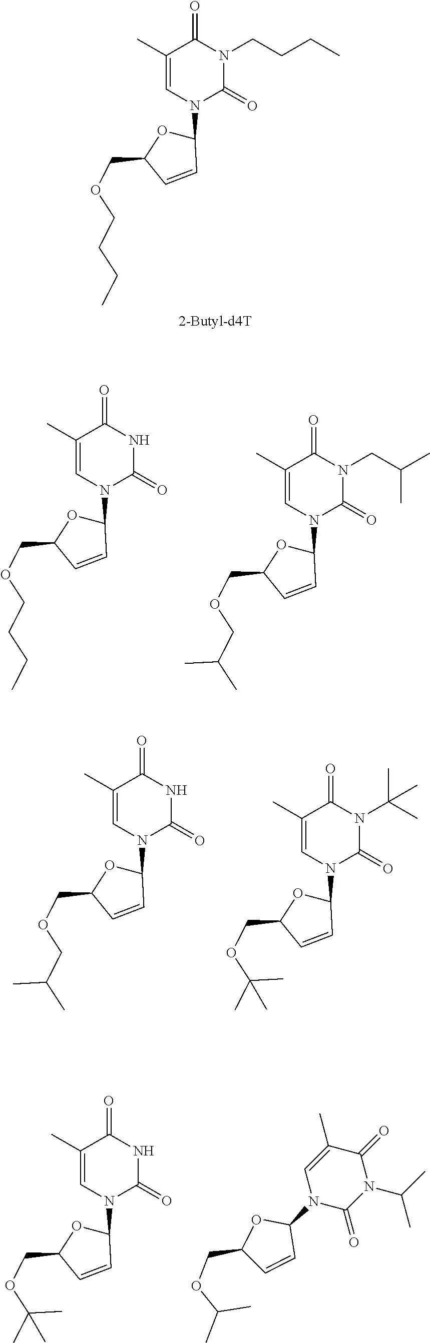

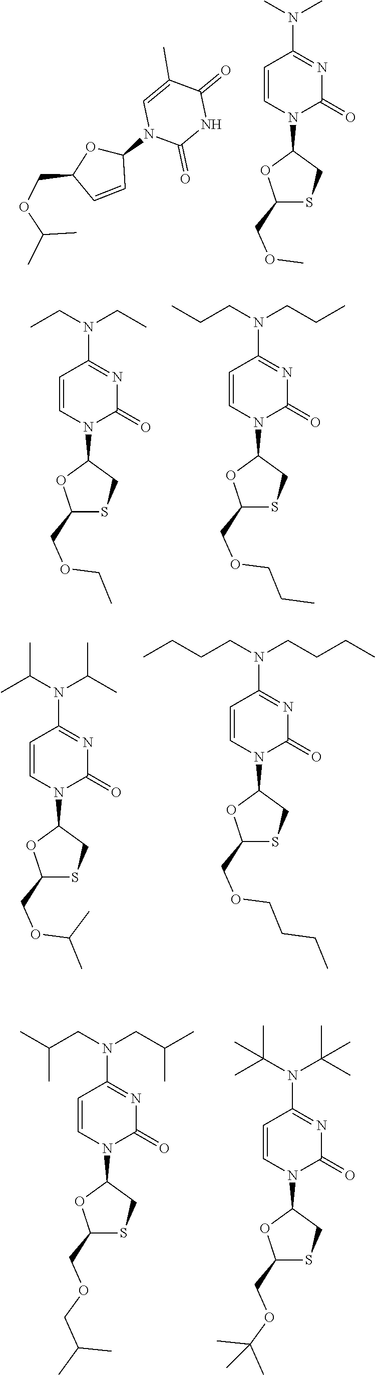

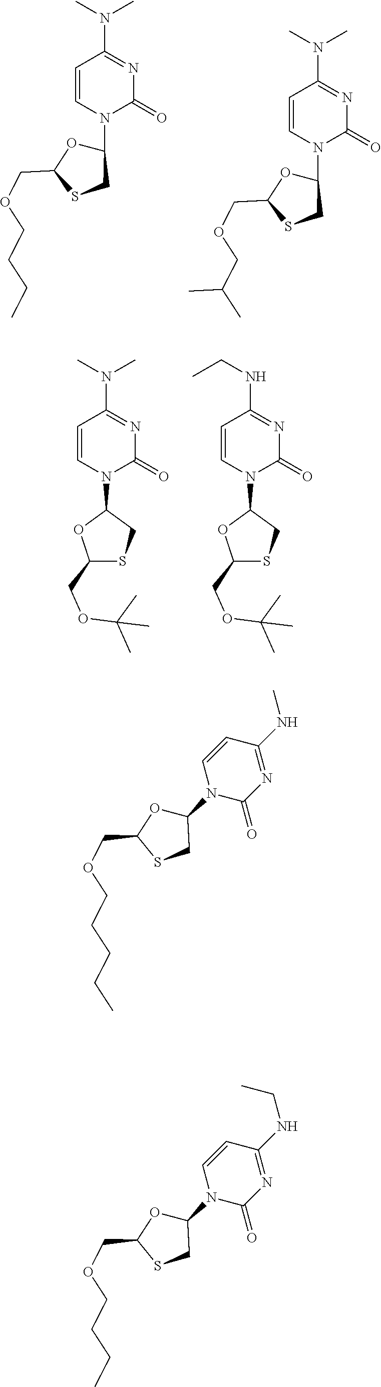

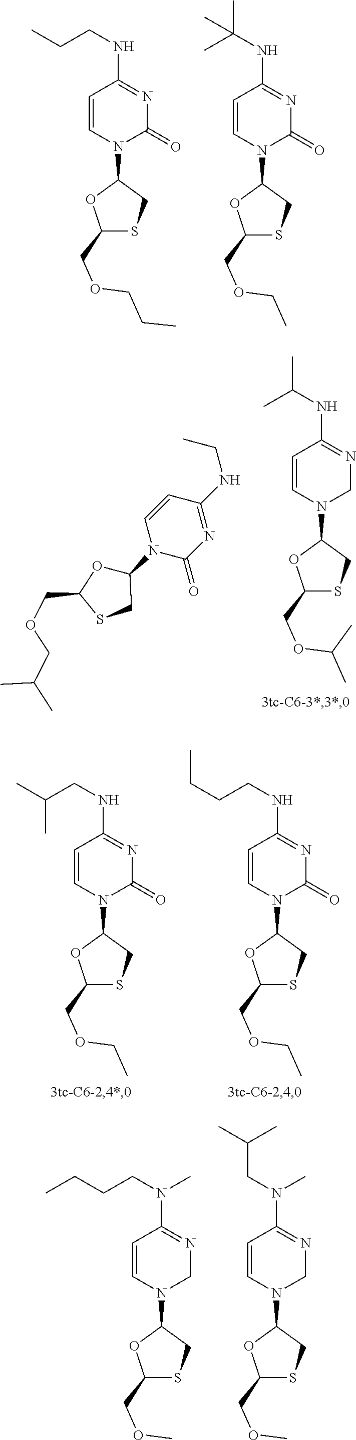

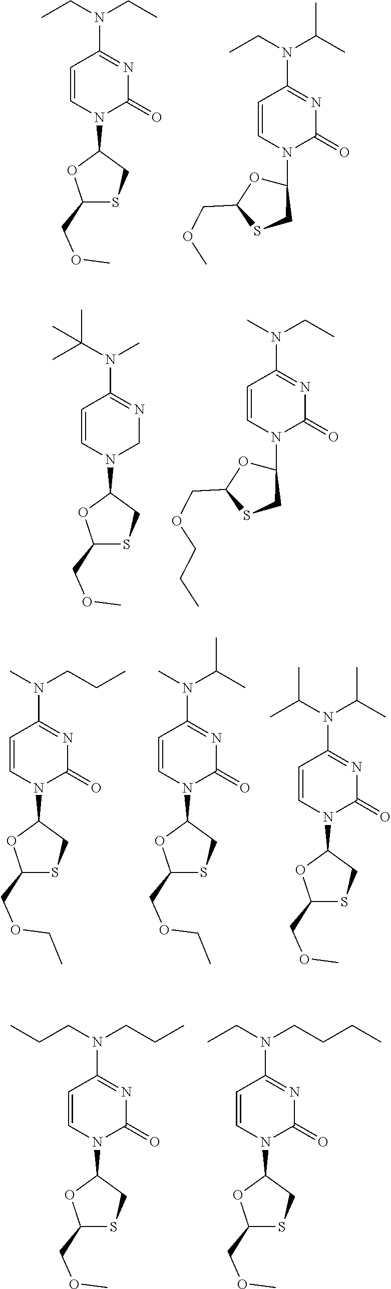

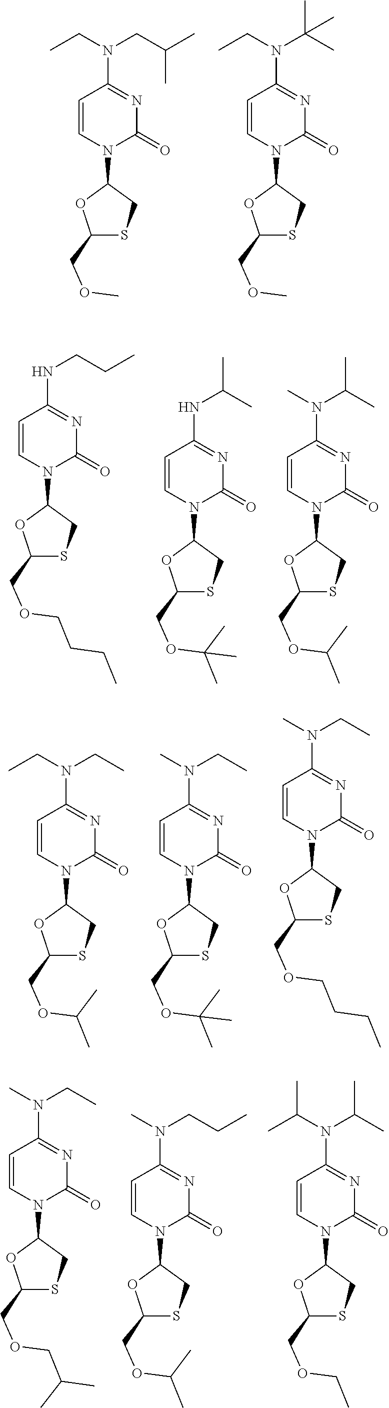

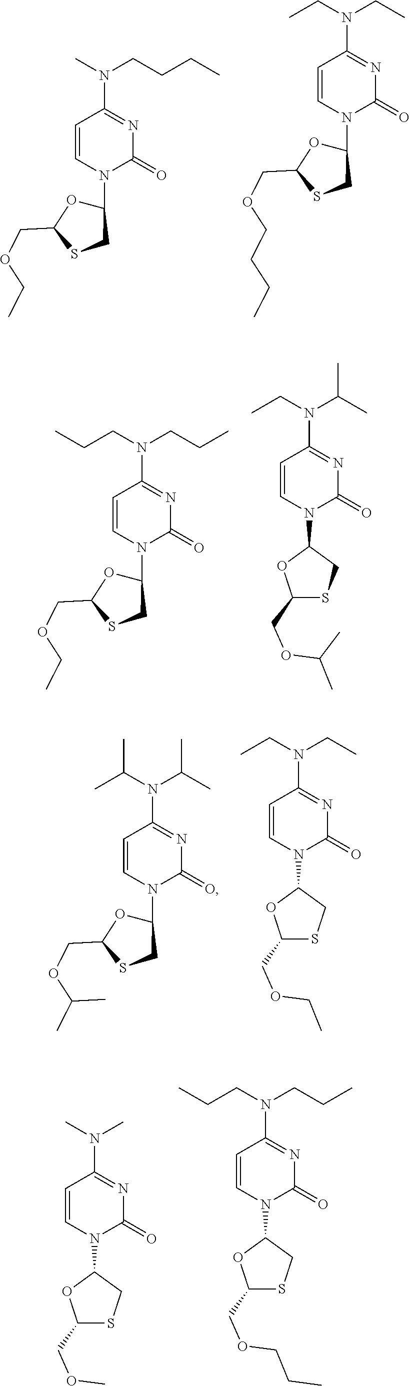

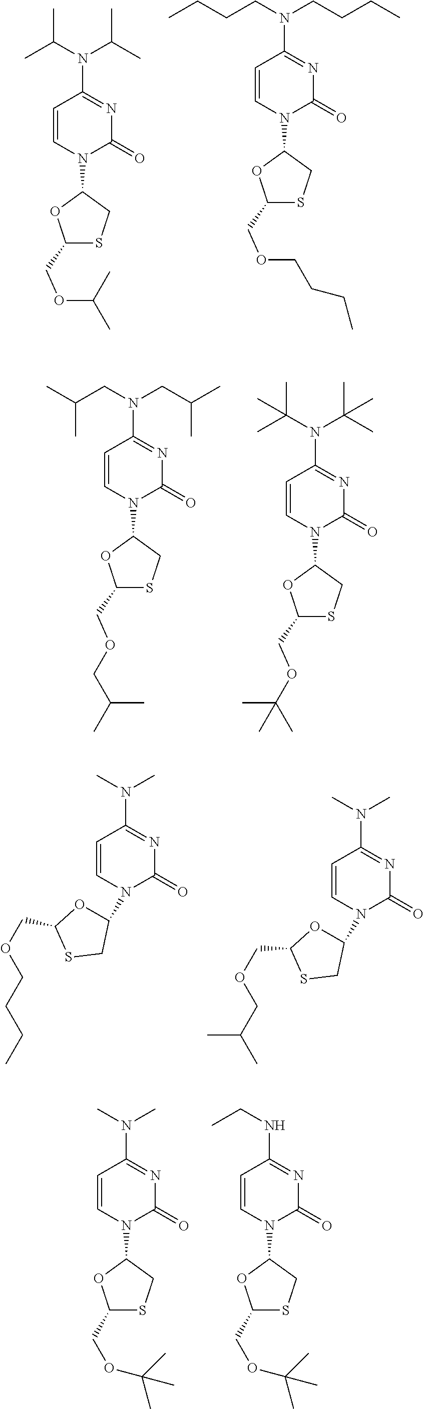

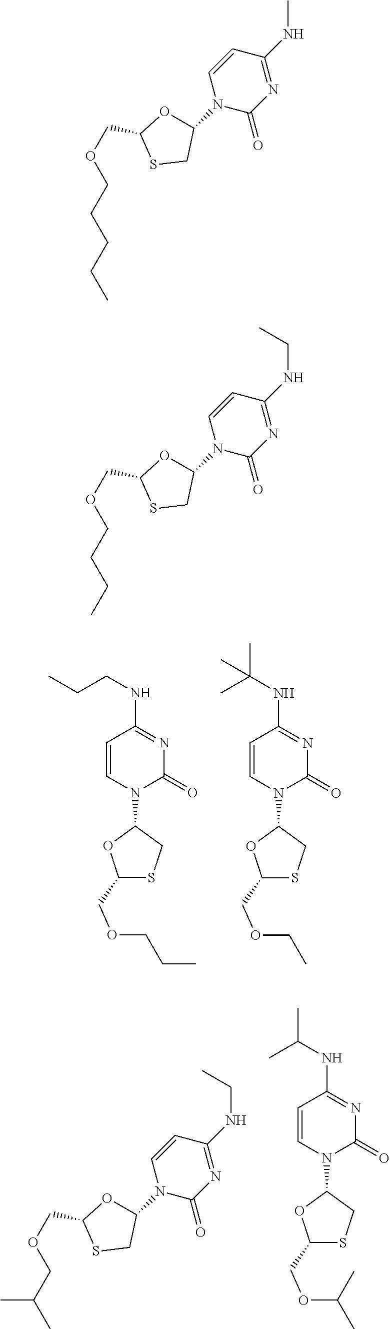

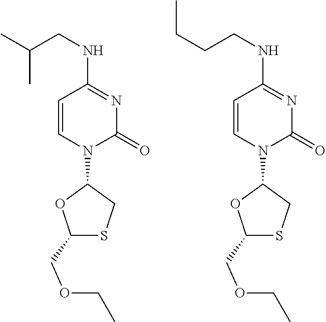 [SMILES: CCOC[C@@H]1O[C@H](n2ccc(NCC(C)C)nc2=O)CS1 ; CCCCNc1ccn([C@@H]2CS[C@H](COCC)O2)c(=O)n1]

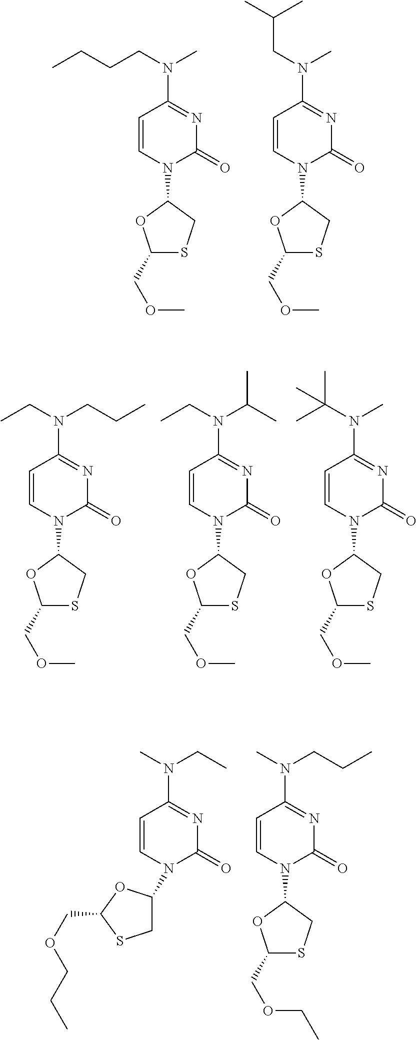

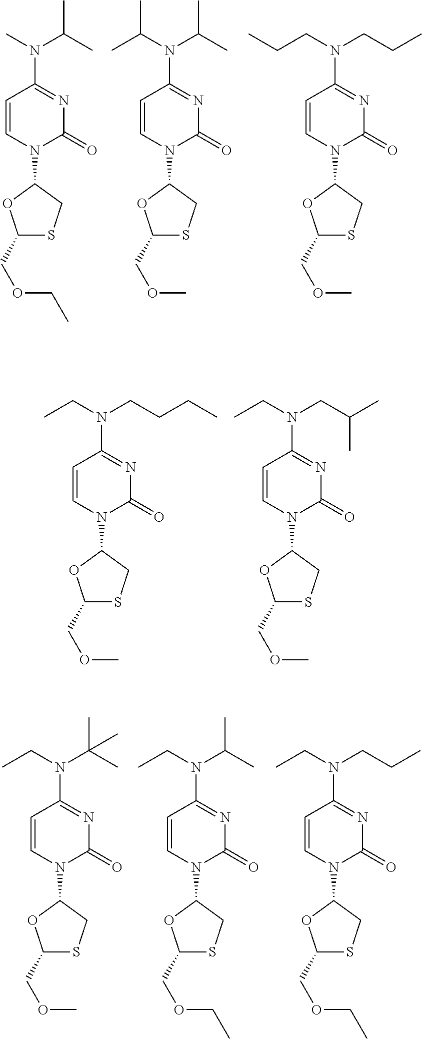

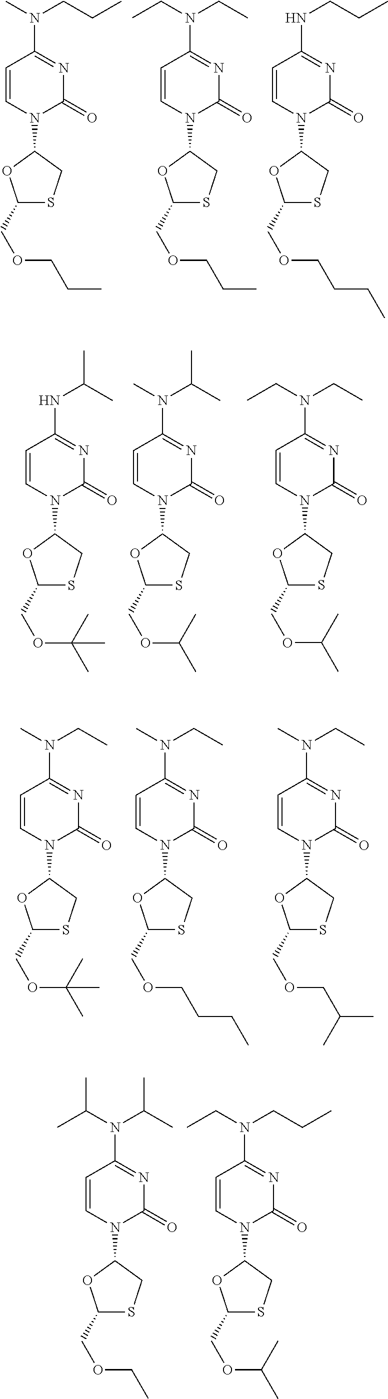

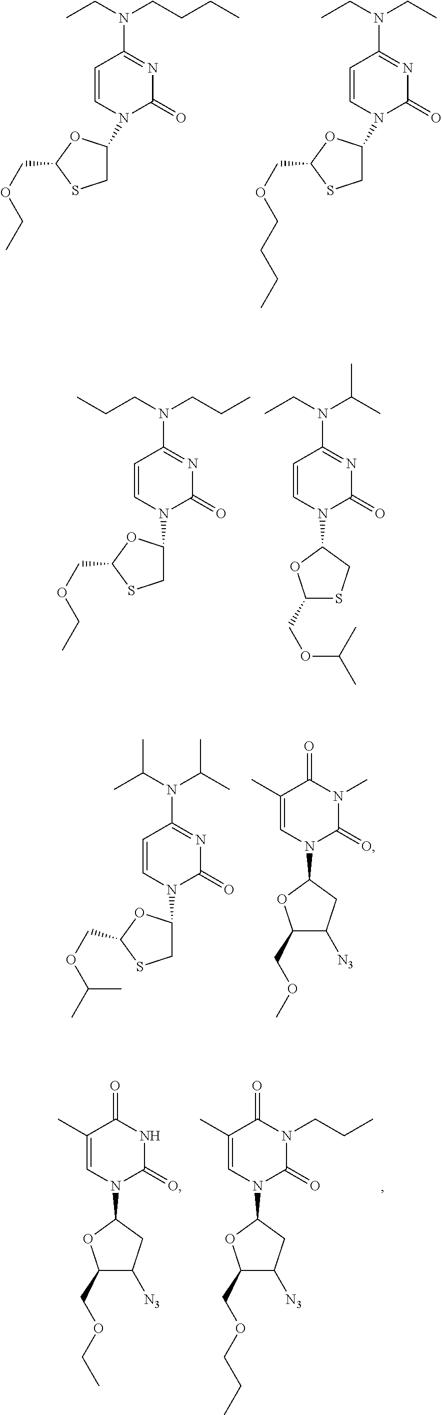

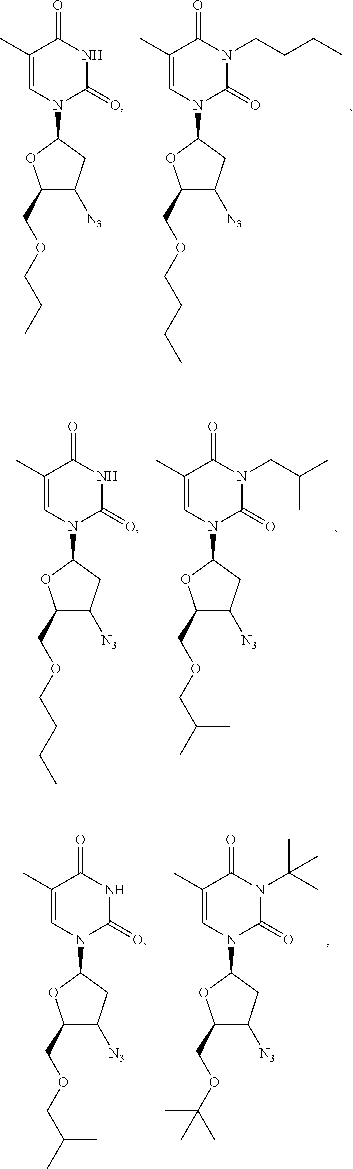 [SMILES: CCCOC[C@H]1O[C@@H](n2cc(C)c(=O)[nH]c2=O)CC1N=[N+]=[N-] ; CCCCOC[C@H]1O[C@@H](n2cc(C)c(=O)n(CCCC)c2=O)CC1N=[N+]=[N-] ; CCCCOC[C@H]1O[C@@H](n2cc(C)c(=O)[nH]c2=O)CC1N=[N+]=[N-] ; Cc1cn([C@H]2CC(N=[N+]=[N-])[C@@H](COCC(C)C)O2)c(=O)n(CC(C)C)c1=O ; Cc1cn([C@H]2CC(N=[N+]=[N-])[C@@H](COCC(C)C)O2)c(=O)[nH]c1=O ; Cc1cn([C@H]2CC(N=[N+]=[N-])[C@@H](COC(C)(C)C)O2)c(=O)n(C(C)(C)C)c1=O]

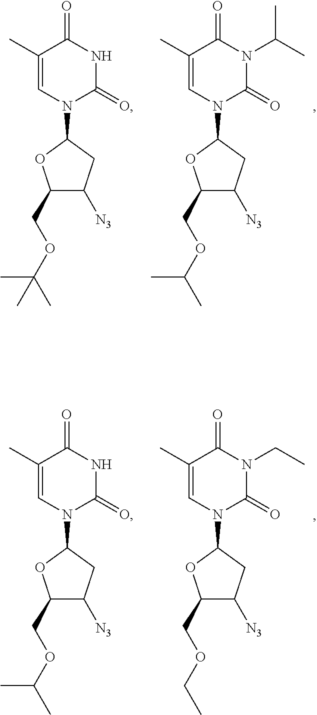 [SMILES: Cc1cn([C@H]2CC(N=[N+]=[N-])[C@@H](COC(C)(C)C)O2)c(=O)[nH]c1=O ; Cc1cn([C@H]2CC(N=[N+]=[N-])[C@@H](COC(C)C)O2)c(=O)n(C(C)C)c1=O ; Cc1cn([C@H]2CC(N=[N+]=[N-])[C@@H](COC(C)C)O2)c(=O)[nH]c1=O ; CCOC[C@H]1O[C@@H](n2cc(C)c(=O)n(CC)c2=O)CC1N=[N+]=[N-]]

an enantiomer or a pharmaceutically acceptable salt thereof.

In one aspect, the vascular retinal disease is diabetic macular edema (DME), branch or central retinal vein occlusion (RVO), neovascular age-related macular degeneration (“wet” AMD), diabetic retinopathy, macular edema due to retinitis pigmentosa, Coats disease, sickle cell disease, polypoidal choroidal vasculopathy or macular neovascularization, either as NRTI or NRTI derivative monotherapy or in combination with anti-VEGF agents. In one aspect, the treatment results in an improvement in visual acuity.

In one aspect, the NRTI or NRTI derivative is administered by intravitreous injection to said subject. In one aspect, the one or more VEGF inhibitors are administered by intravitreous injection to said subject either before, after or at the same time as administration of the NRTI or NRTI derivative.

### BRIEF DESCRIPTION OF THE DRAWINGS

FIG. 1A-1B. Mean Change in Visual Acuity over Time. (A) Results for Early Treatment of Diabetic Retinopathy Study (ETDRS) best corrected Visual-Acuity Letter Score (VALS) in Randomized Clinical Trial of Lamivudine versus Placebo. (B) Results for ETDRS best corrected VALS including synthetic controls from the DRCR.net Protocol T Study.

FIG. 2. Percentage of Eyes Gaining 15 or More in Visual-Acuity Letter Score Including Synthetic Controls from DRCR.net Protocol T. Improvement in VALS of 15 or more (equivalent to 3 lines on Snellen eye chart) including synthetic controls from the DRCR.net Protocol T Study.

FIG. 3. Mean Change in Visual Acuity Over Time Including Synthetic Controls from DRCR.net Protocol T-Propensity Score Matched Model. Results for ETDRS best corrected VALS using a propensity score matched model including synthetic controls from the DRCR.net Protocol T Study.

FIG. 4. Mean Change in Central Subfield Thickness over Time Including Synthetic Controls from DRCR.net Protocol T. Results for retinal central subfield thickness, measured by optical coherence tomography, including synthetic controls from the DRCR.net Protocol T Study.

FIGS. 5A-5D. K-9 inhibits RRD-induced caspase-1 cleavage. (A) Representative immunoblot; retinal pro-caspase-1 and caspase-1 in RRD model at baseline, 24-hrs, 48-hrs, and 72-hrs post-SRI (3 μl 1% CMC; WT mice; n=3). (B) Densitometric quantification of caspase-1 from FIG. 1A, normalized to GAPDH (two-tailed t-test; *P<0.05; **P<0.01; n=3; shown as mean±SEM). (C) Representative immunoblot; day 3 retinal pro-caspase-1 and caspase-1 in RRD model treated with PBS, K-9L, or K-9H (n=3). (D) Densitometric quantification of caspase-1 from FIG. 1C, normalized to GAPDH (two-tailed t-test; *P<0.05; ***P<0.001, n=3; shown as mean±SEM).

FIGS. 6A-6B. NRTIs inhibit RRD-induced caspase-1 cleavage. (A) Representative immunoblot; day 3 retinal pro-caspase-1 and caspase-1 in RRD model after IP PBS, 3TC, or AZT (n=3). (B) Densitometric quantification of caspase-1 from FIG. 2A normalized to GAPDH (Student's two-tailed t-test; *P<0.05; **P<0.01; ***P<0.001; n=3; shown as mean±SEM).

FIGS. 7A-7B. K-9 protects photoreceptors in RRD model. (A) Representative confocal photographs of retinal ONL from day 3 RRD models treated with PBS, K-9L, or K-9H (n=12-14 per group); stained with TUNEL (above; green) and TUNEL/DAPI (below; green and blue). Scale bars represent 100 μm. (B) Quantification of TUNEL-positive ONL cells (Student's two-tailed t-test; **P<0.01; ****P<0.0001; n=12-14 per group; data represented as mean±SEM).

FIGS. 8A-8B. NRTIs attenuate photoreceptor death in RRD model. (A) Representative confocal photographs of retinal ONL from day −3 RRD models treated with 3TC, AZT, or PBS (n=12-14 per group); stained with TUNEL (above; green) or TUNEL/DAPI (below; green and blue). Scale bars represent 100 μm. (B) Quantification of day −3 TUNEL-positive ONL cells (Student's two-tailed t-test; **P<0.01; ****P<0.0001; n=12-14 per group; data represented as mean±SEM).

FIG. 9. PBS injection induced a mild reduction in a-wave and b-wave amplitudes. VEGF injection a substantial reduction in a-wave and b-wave amplitudes. K8 IVt injection markedly improved the a-wave and b-wave amplitudes. 3TC, K9, and AZT IP injections markedly improved the a-wave and b-wave amplitudes.

FIG. 10. Describes Study Participants: 24 participants from a single, tertiary center who completed the 8-week duration of the trial, were randomly assigned to receive oral lamivudine (10 participants; 16 eyes) or placebo (14 participants; 21 eyes) (Table 2 as shown in FIG. 10).

### DETAILED DESCRIPTION OF THE INVENTION

Diabetes Mellitus (DM) is a global epidemic that in the coming decades may affect approximately 700 million people (1). The increasing prevalence of DM will toll a higher incidence of vascular complications such as diabetic retinopathy, a principal cause of blindness in economically active adults. Proliferative diabetic retinopathy and diabetic macular edema (DME) are the two complications responsible for vision loss in diabetics. Between 2005 and 2008, 4.4% of the population or 1.8 million people in the United States had one of the two complications. With the increasing prevalence of DM, ophthalmologic complications will still remain a serious public health problem well into the future.

Macular edema refers to accumulation of fluid in the extracellular space of the central retina. Specifically, in DME, edema is related to the breakdown of the internal and external blood-retinal barrier caused by increased secretion of growth factors and inflammatory cytokines that alter the junctions between endothelial cells and the retinal pigment epithelium (RPE). The inflammatory process is one of the initial events in the pathophysiology of diabetic retinopathy; the hyperglycemic state induces the production of adhesion molecules in endothelial cells and circulating monocytes, which causes capillary occlusion and intracellular fluid leakage. The interaction between endothelial cells and monocytes followed by the migration of these monocytes to the retinal extracellular space triggers the production of pro-antigenic and pro-inflammatory factors. Other cell types such as Muller cells, a type of glial cell, respond to these factors and also contribute to potentiate this vicious cycle. Vascular endothelial growth factor (VEGF) and cytokines such as tumor necrosis factor (TNF)-α induce loss of intercellular junctions and induce vascular leakage. Interleukin (IL)-1β and IL-6 have already been identified in the vitreous of patients with advanced diabetic retinopathy and are elevated in animal models of increased vascular permeability, a central feature of DME.

First-line treatment for DME aims to neutralize one or more of these angio-inflammatory factors and consists of intravitreal injections of anti-VEGF or corticosteroids. Despite the undeniable evolution in the treatment of DME provided by these drugs, about 40% of treated patients remain with residual edema at the end of treatment, regardless of the protocol used (9). Intraocular injections are not without risks and include common complications such as the incidence of cataracts after steroid injection or rarer complications, but with potential for serious sequelae, such as glaucoma and intraocular infection (endophthalmitis). In addition, the number of injections recommended by clinical studies to keep the disease under control generates high costs for the patient and decreases treatment adherence, which reduces the effectiveness of the treatment when compared to large prospective clinical studies.

Apart from microvasculopathy, which promotes retinal edema, neurodegeneration is an independent pathologic feature of diabetic retinopathy. Retinal hypoxia is another edema-independent etiology of retinal dysfunction and vision loss in diabetic retinopathy. In addition to intraretinal fluid that is commonplace in DME, subretinal fluid also can accompany DME and can exacerbate retinal hypoxia by impeding oxygen transport due to increased diffusion distance from choroidal vasculature to the retina. Therefore, therapies that address hypoxia and neurodegeneration in addition to microvasculopathy and edema could be beneficial in improving vision in patients with DME.

In an attempt to address the points raised above, new therapeutic targets are under study. The ideal target should cause a more efficient and extensive blockage of the inflammatory process and decrease the recruitment of inflammatory cells to the retina. A great deal of effort has also been made to identify treatments that can be given orally instead of ocular injections, which would remove one of the major barriers to treating DME. Among the candidates, there is a molecular complex of the innate immune system called inflammasome. This complex has been implicated in the pathophysiology of many chronic inflammatory diseases such as DM, systemic lupus erythematosus and Alzheimer's disease. Studies in retinal diseases such as geographic atrophy and proliferative diabetic retinopathy also suggest that increased inflammasome activity may play an important role in the pathogenesis of these diseases.

Inflammasomes are members of a large family of multimeric proteins capable of recognizing different endogenous and exogenous pathogenic molecules. When activated by one of these molecules, the inflammasome initiates the production of pro-inflammatory cytokines (IL-1β and IL-18) which, in turn, induces cell death. The NLRP3 inflammasome is the most studied member of this family and has been identified in several cells relevant to the pathogenesis of diabetic retinopathy, such as retinal pigment epithelium cells (RPE), Muller cells, microglia, macrophages and endothelial cells. It is noteworthy that preclinical studies in models analogous to diabetic retinopathy and DME suggest that the pharmacological blockade of NLRP3 reduces retinal vascular permeability and significantly improves the disease phenotype, making it an attractive option to be explored in clinical studies.

The class of nucleotide reverse transcriptase inhibitors (NRTIs) is one of the pillars in the treatment of infection by the human immunodeficiency virus (HIV) and hepatitis B virus (HBV). In addition, this class has anti-inflammatory activity thanks to its intrinsic ability to inhibit inflammasome activation. Based on preclinical and retrospective clinical studies, the expansion of the clinical use of NRTIs has been suggested for different chronic systemic diseases such as DM, in addition to ocular diseases such as geographic atrophy, choroidal neovascularization and diabetic retinopathy.

Among the many NRTIs, Lamivudine (3TC), has well-established pharmacodynamics and pharmacokinetics for over three decades and has a low drug interaction rate, and thus is approved for long-term use in life-threatening infections such as HIV or hepatitis B. Among the side effects of this class of drugs, the potential for mitochondrial toxicity of NRTIs stands out, which can lead to lactic acidosis in rare cases. However, newer versions of NRTIs, such as 3TC, have a reduced incidence of lactic acidosis when compared to earlier generation drugs in the same class.

The expansion of the use of drugs already approved by regulatory agencies is a known and beneficial strategy as it reduces costs and time to access new therapies. Provided herein is the efficacy of 3TC in HIV-negative and hepatitis B-negative patients with DME.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R. J. Lewis, John Wiley & Sons, New York, N.Y., 2001.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

The term “about” can refer to a variation of +5%, +10%, +20%, or +25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group.

Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “physiologically functional derivative” means any pharmaceutically acceptable derivative of a compound of the present disclosure. For example, an amide or ester of a compound which upon administration to a subject, particularly a mammal, is capable of providing, either directly or indirectly, a compound of the present disclosure of an active metabolite thereof.

The terms “treatment” or “treating” refer to the medical management of a subject with the intent to cure, ameliorate, stabilize, or prevent a condition or disorder (e.g., retinal degradation). This term includes active treatment, that is, treatment directed specifically toward the improvement of a condition, and also includes causal treatment, that is, treatment directed toward removal of the cause of the associated condition. In addition, this term includes palliative treatment, that is, treatment designed for the relief of symptoms rather than the curing of the condition; preventative treatment, that is, treatment directed to minimizing or partially or completely inhibiting the development of symptoms or disorders of the associated condition; and supportive treatment, that is, treatment employed to supplement another specific therapy directed toward the improvement of the associated disease, pathological condition, or disorder.

With regard to administering the compound, the term “administering” refers to any method of providing a composition and/or pharmaceutical composition thereof to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, subcutaneous administration, intra vitreous administration, including via intravitreous sustained drug delivery device, intracameral (into anterior chamber) administration, suprachoroidal injection, subretinal administration, subconjunctival injection, sub-Tenon's administration, peribulbar administration, transscleral drug delivery, administration via topical eye drops, and the like. Administration can be continuous or intermittent. In various aspects, a preparation can be administered therapeutically; that is, administered to treat an existing disease or condition (e.g., exposure to OP compounds). In further various aspects, a preparation can be administered prophylactically; that is, administered for prevention of a disease or condition.

The term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition. For example, for oral administration a dose or doses of up to about 1200 mg per day or up to about 600 mg per day or up to about 300 mg per day or up to about 150 mg per day or up to about 50 mg per day can be administered. For example, for intra-vitreous administration a dose or doses of up to about 1 mg or up to about 0.5 mg or up to about 0.25 mg or up to 0.1 about mg can be administered.

The terms “subject” or “subject in need thereof refer to a target of administration, which optionally displays symptoms related to a particular disease, condition, disorder, or the like. The subject(s) of the herein disclosed methods can be human or non-human (e.g., primate, horse, pig, rabbit, dog, sheep, goat, cow, cat, guinea pig, rodent, and non-mammals). The term “subject” does not denote a particular age or sex. Thus, adult and newborn subjects, as well as fetuses, whether male or female, are intended to be covered. The term “subject” includes human and veterinary subjects.

As will be recognized by one of ordinary skill in the art, the terms “suppression,” “suppressing,” “suppressor,” “inhibition,” “inhibiting” or “inhibitor” do not refer to a complete elimination in all cases. Rather, the skilled artisan will understand that the term “suppressing” or “inhibiting” refers to a reduction or decrease. Such reduction or decrease can be determined relative to a control. In some embodiments, the reduction or decrease relative to a control can be about a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% decrease.

In some exemplary embodiments, the presently disclosed subject matter includes methods for treating retinal detachment. Some methods of the present disclosure comprise administering to a subject in need thereof an effective amount of a composition for treating retinal detachment.

As described herein, the presently disclosed subject matter further includes pharmaceutical compositions comprising the compounds described herein together with a pharmaceutically acceptable carrier.

The term “pharmaceutically acceptable carrier” refers to sterile aqueous or nonaqueous solutions, dispersions, suspensions or emulsions, as well as sterile powders for reconstitution into sterile injectable solutions or dispersions just prior to use. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

Suitable formulations include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The formulations can be presented in unit-dose or multi-dose containers, for example sealed ampoules and vials, and can be stored in a frozen or freeze-dried (lyophilized) condition requiring only the addition of sterile liquid carrier immediately prior to use.

For oral administration, the compositions can take the form of, for example, tablets or capsules prepared by a conventional technique with pharmaceutically acceptable excipients such as binding agents {e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycol late); or wetting agents (e.g., sodium lauryl sulphate). The tablets can be coated by methods known in the art.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration the compositions can take the form of tablets or lozenges formulated in conventional manner.

The compositions can be formulated as eye drops. For example, the pharmaceutically acceptable carrier may comprise saline solution or other substances used to formulate eye drop, optionally with other agents. Thus, eye drop formulations permit for topical administration directly to the eye of a subject.

The compositions can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). The compounds can also be formulated in rectal compositions, creams or lotions, or transdermal patches.

The presently disclosed subject matter further includes a kit that can include a compound or pharmaceutical composition as described herein, packaged together with a device useful for administration of the compound or composition. As will be recognized by those or ordinary skill in the art, the appropriate administration-aiding device will depend on the formulation of the compound or composition that is selected and/or the desired administration site. For example, if the formulation of the compound or composition is appropriate for injection in a subject, the device could be a syringe. For another example, if the desired administration site is cell culture media, the device could be a sterile pipette.

Provided herein are nucleoside reverse transcriptase inhibitors (NRTIs) and derivatives alone or in combination with VEGF (Vascular endothelial growth factor) inhibitors for the treatment of retinal vascular disorders. Vascular endothelial growth factor (VEGF) inhibitors have been shown to reduce retinal edema (thickening) and improve vision in diabetic macular edema (DME), retinal vein occlusion (RVO), and neovascular age-related macular degeneration (“wet” AMD), collectively termed ocular disorders. Nucleoside reverse transcriptase inhibitors (NRTIs) and alkylated derivatives of NRTIs (Kamuvudines) block inflammasome activation and confer neuroprotection. Methods of treating ocular disorders and improving vision using NRTIs or NRTI derivatives either in isolation or in combination with VEGF inhibitors are provided.

The methods provided herein comprise administration of one or more compounds of structural Formula (I)

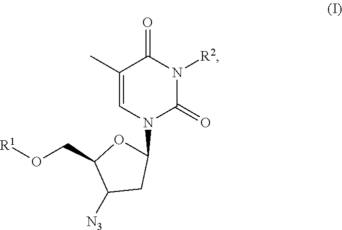

wherein:
R¹ is C₁₋₄ alkyl; and
R² is H or C₁₋₄ alkyl, provided that when R² is H, R¹ is not CH₃ or a pharmaceutically salt thereof and optionally present in a carrier. In some embodiments, R² is CH₃ or CH₂CH₃. In other embodiments, R¹ is n-C₄H₉.

As used herein the term “alkyl” refers to C₁₋₄ inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, and/or tert-butyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₄ straight-chain alkyls. In other embodiments, “alkyl” refers, in particular, to C₁₋₄ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The methods provided herein also comprise administration of one or more compounds including:

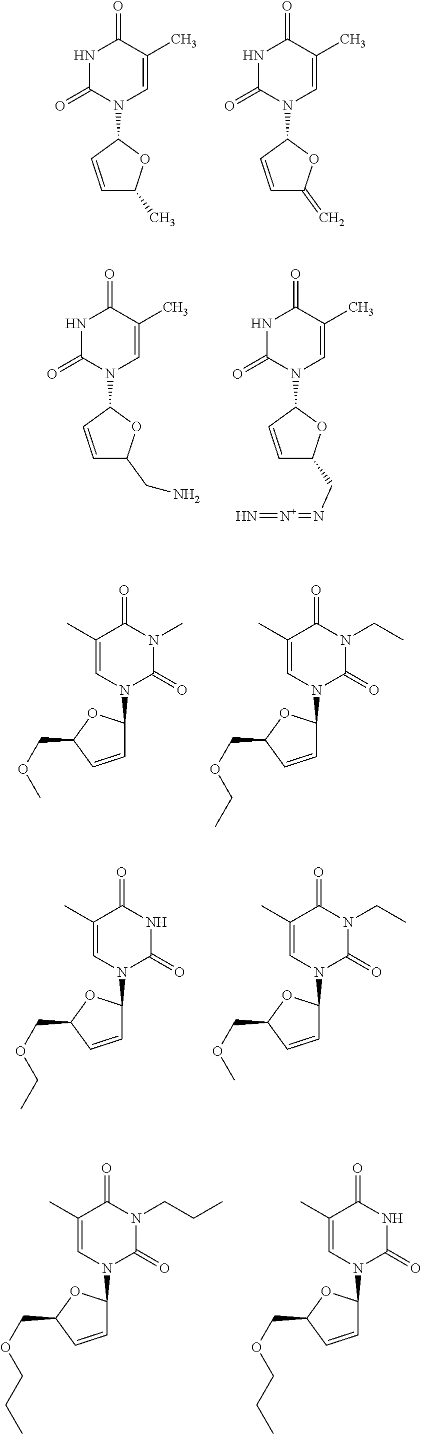

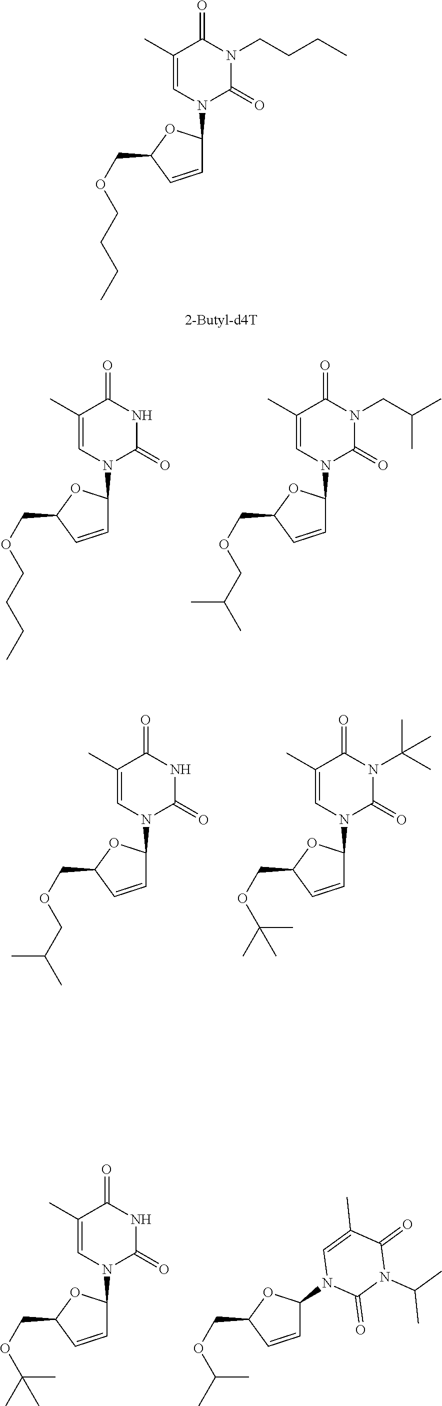

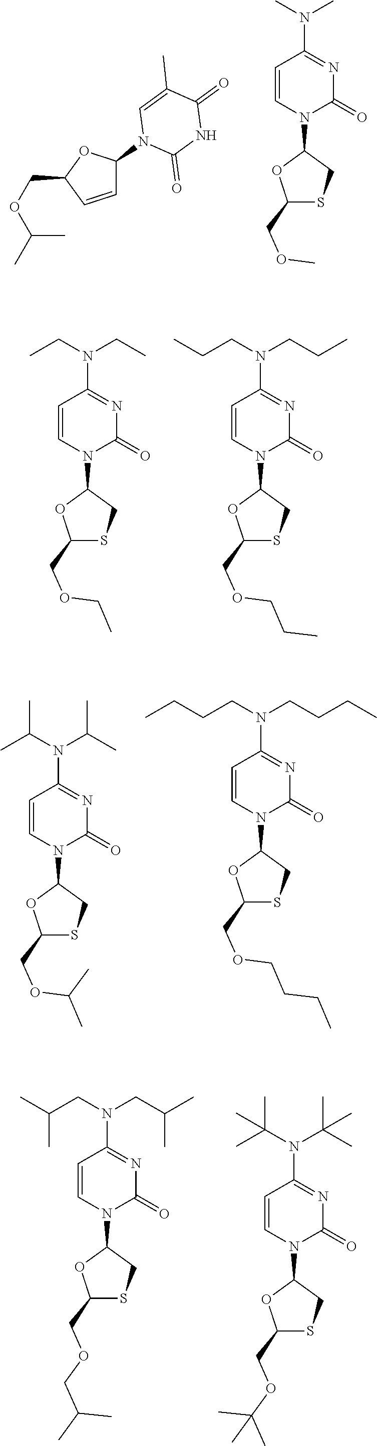

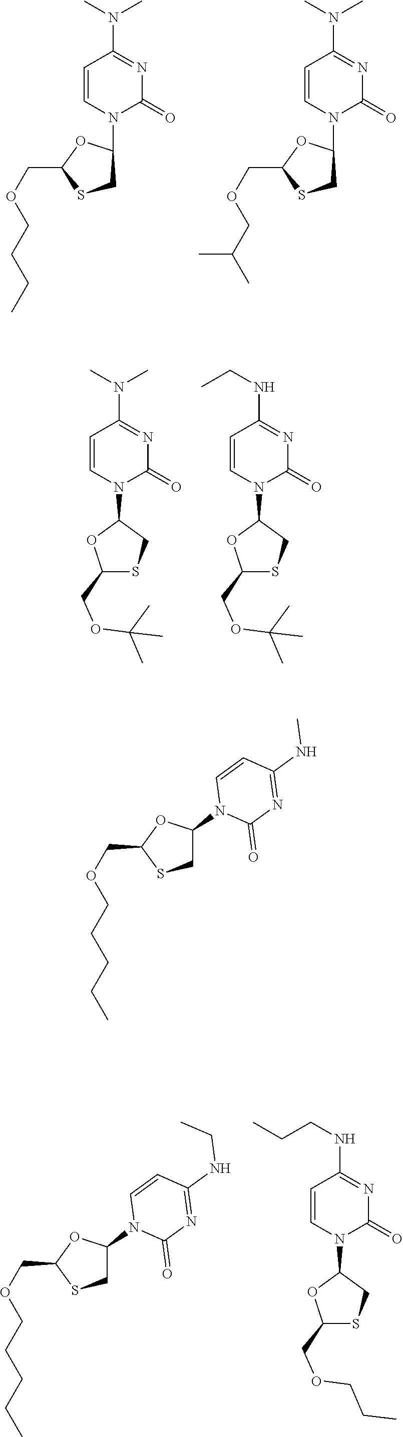

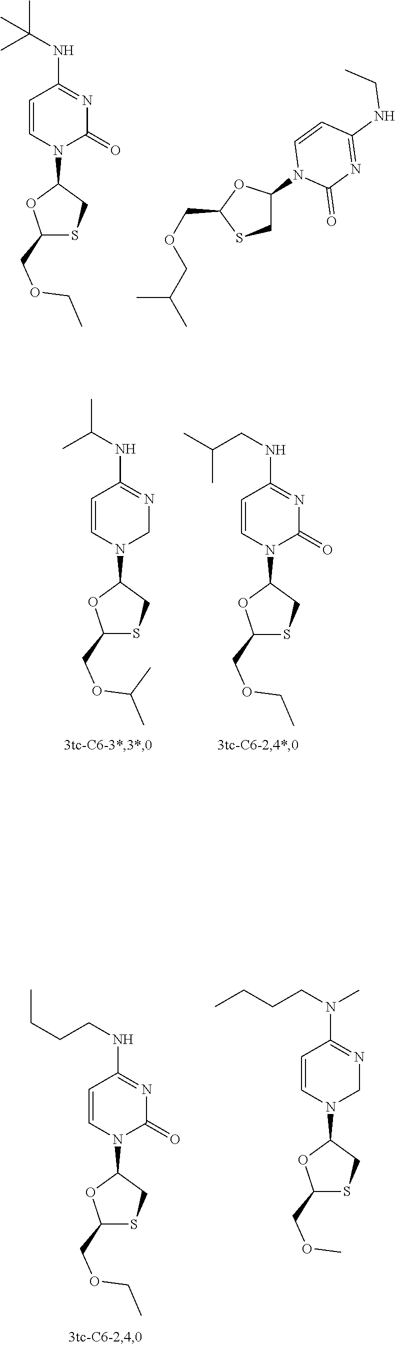

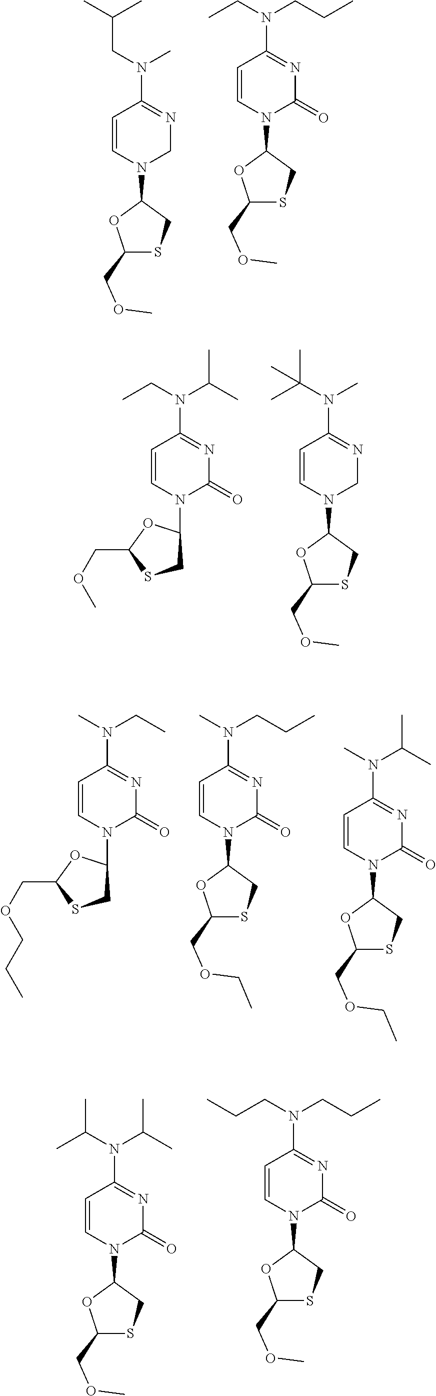

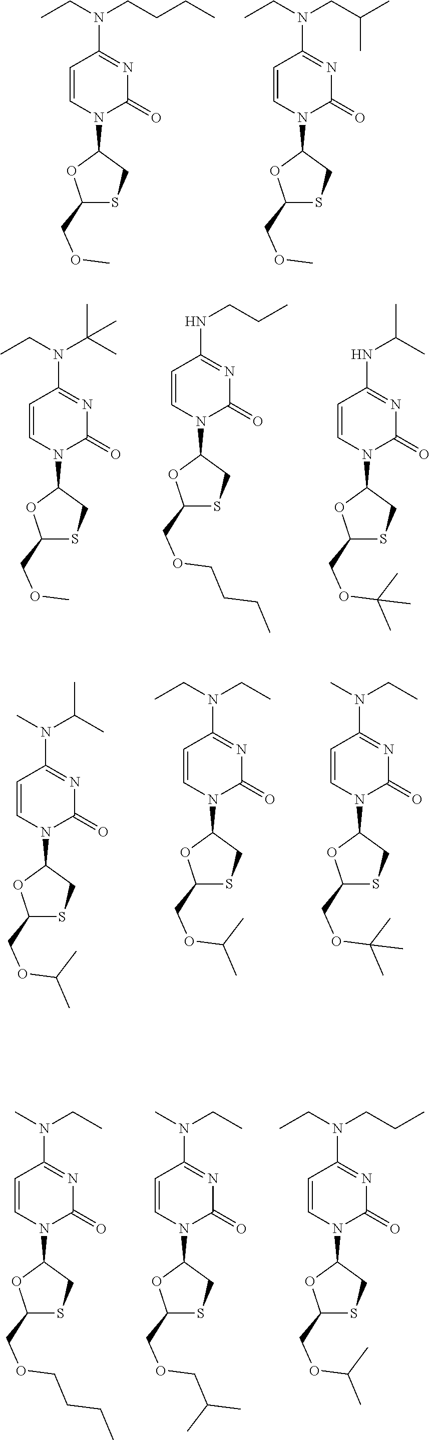

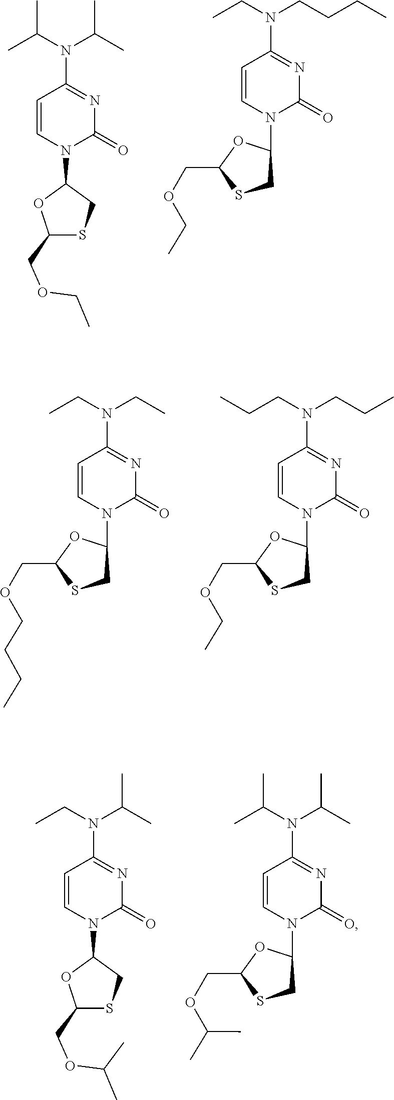

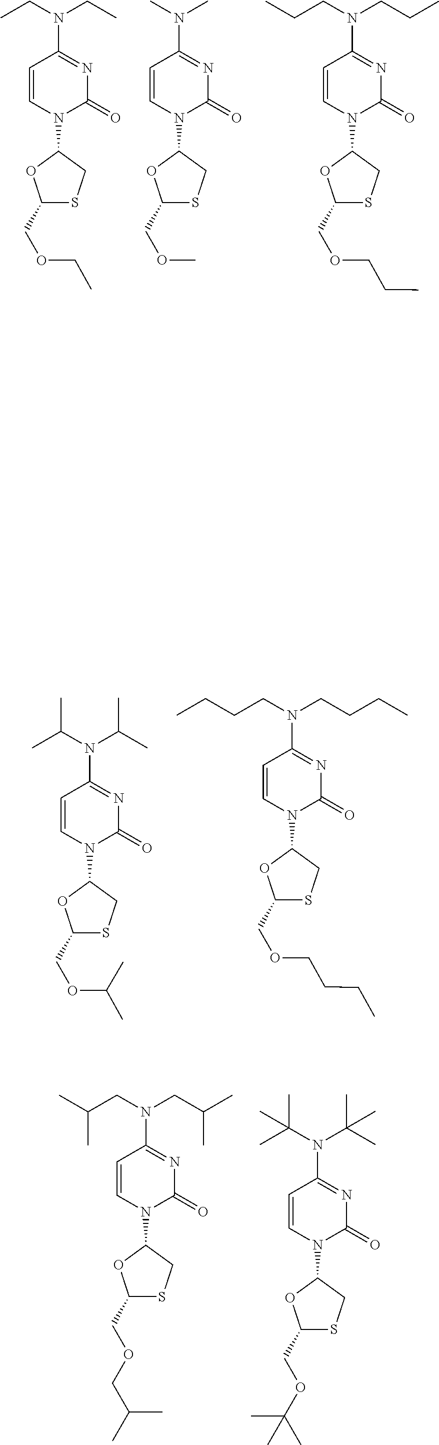

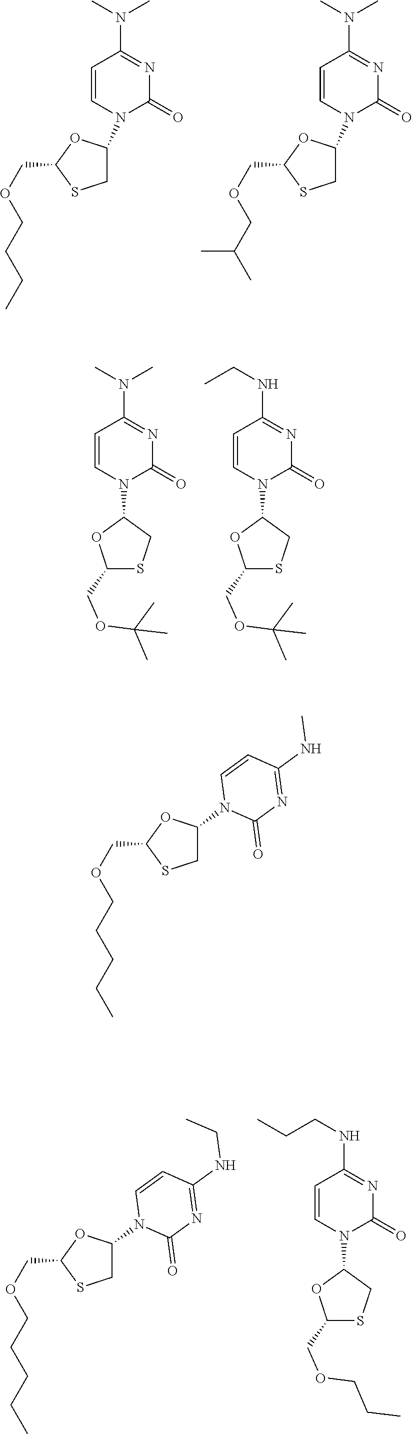

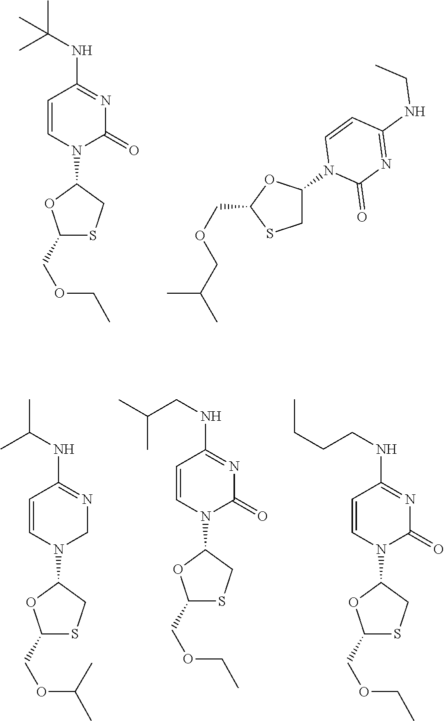 [SMILES: CCOC[C@@H]1O[C@H](n2ccc(NC(C)(C)C)nc2=O)CS1 ; CCNc1ccn([C@@H]2CS[C@H](COCC(C)C)O2)c(=O)n1 ; CC(C)NC1=NCN([C@@H]2CS[C@H](COC(C)C)O2)C=C1 ; CCOC[C@@H]1O[C@H](n2ccc(NCC(C)C)nc2=O)CS1 ; CCCCNc1ccn([C@@H]2CS[C@H](COCC)O2)c(=O)n1]

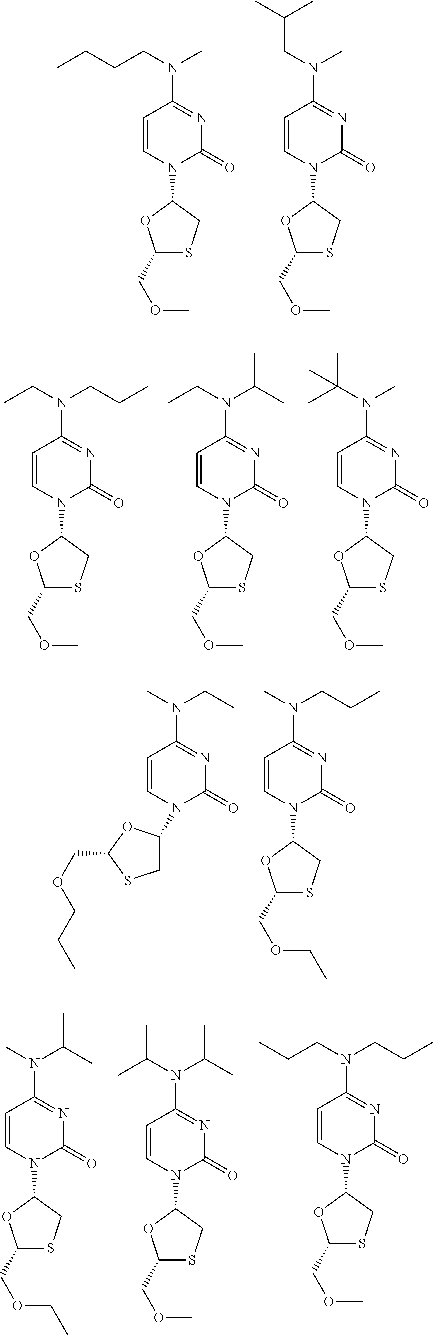

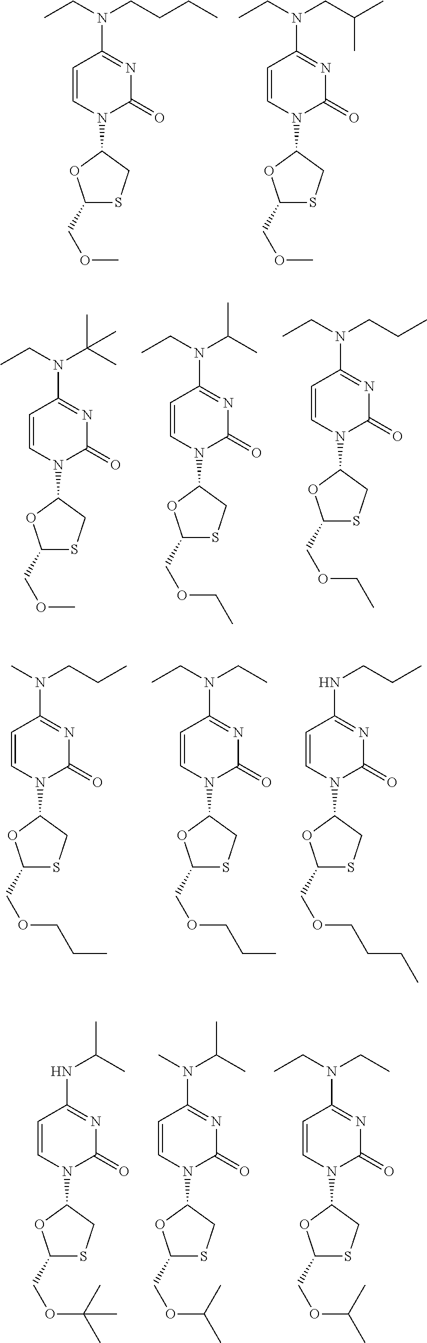

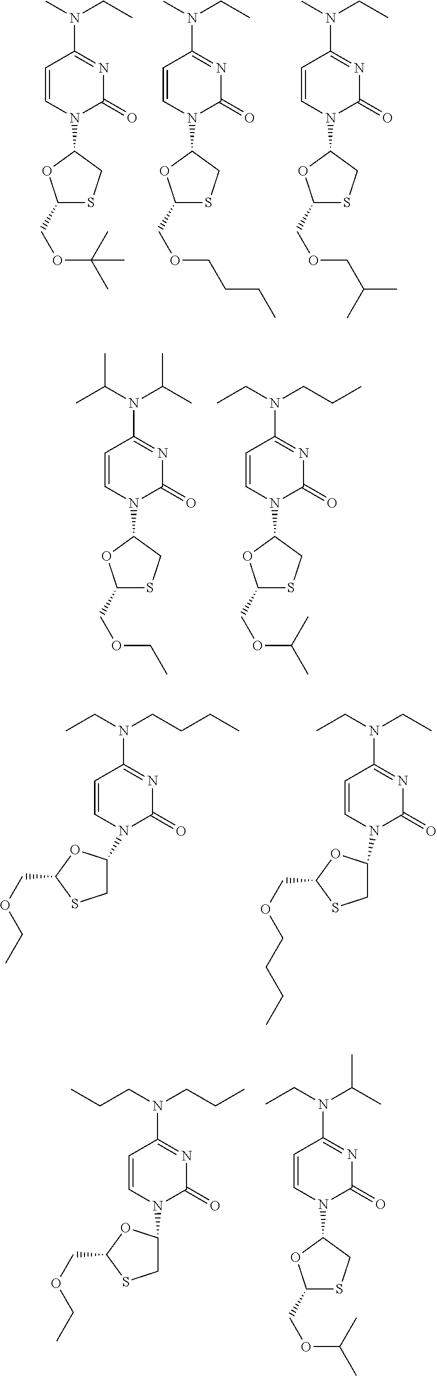

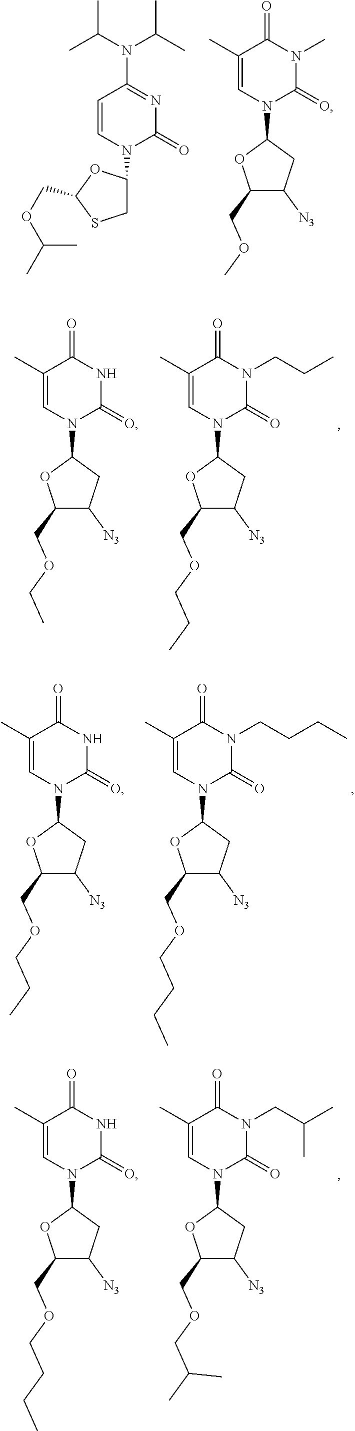

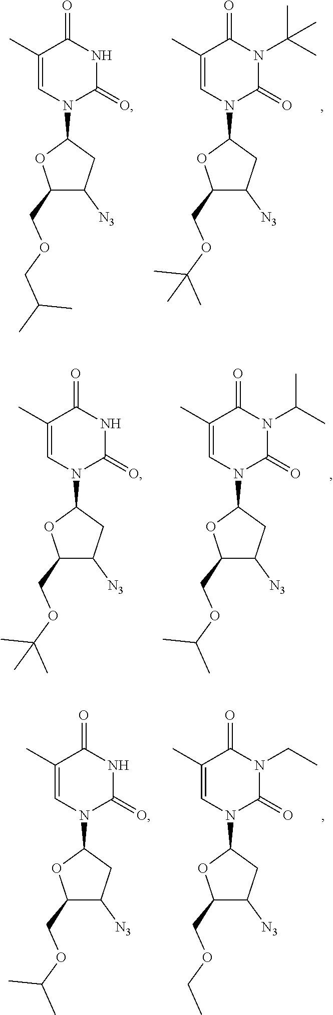 [SMILES: Cc1cn([C@H]2CC(N=[N+]=[N-])[C@@H](COCC(C)C)O2)c(=O)[nH]c1=O ; Cc1cn([C@H]2CC(N=[N+]=[N-])[C@@H](COC(C)(C)C)O2)c(=O)n(C(C)(C)C)c1=O ; Cc1cn([C@H]2CC(N=[N+]=[N-])[C@@H](COC(C)(C)C)O2)c(=O)[nH]c1=O ; Cc1cn([C@H]2CC(N=[N+]=[N-])[C@@H](COC(C)C)O2)c(=O)n(C(C)C)c1=O ; Cc1cn([C@H]2CC(N=[N+]=[N-])[C@@H](COC(C)C)O2)c(=O)[nH]c1=O ; CCOC[C@H]1O[C@@H](n2cc(C)c(=O)n(CC)c2=O)CC1N=[N+]=[N-]]

an enantiomer or a pharmaceutically acceptable salt thereof.

The details of one or more embodiments of the presently disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently disclosed subject matter is further illustrated by the following specific but non-limiting examples.

### Example I

### Effect of Oral Lamivudine Versus Placebo in Diabetic Macular Edema: A Randomized Clinical Trial

### Introduction

Diabetic macular edema (DME) is the most common cause of vision loss in diabetic retinopathy, which is prevalent in over 25% of people with diabetes and affects nearly 10 million individuals in the United States.¹ With the rising prevalence of diabetes, which affects over 10% of adults worldwide,² vision loss from and associated health care costs of treating DME are important global health issues.

The standard of care for DME, particularly for eyes with poor visual acuity, is monthly intravitreous injections of anti-vasopermeability drugs, including inhibitors of vascular endothelial growth factor (VEGF) such as aflibercept, bevacizumab, and ranibizumab.^{3,4} On the basis of Medicare allowable charges, the monthly cost of these drugs (aflibercept 2 mg, $1,751; ranibizumab 0.5 mg, $1,181) and administering them via intravitreous injection (up to $143) is substantial. Also, intravitreous injection of these drugs carries risks of intraocular infection (approximately 1 in 3,000) and retinal detachment (approximately 1 in 7,500).^{5,6} With tens of millions of intravitreous injections performed globally every year,⁷ tens of thousands of people develop these vision-threatening complications annually. In addition, monthly visits to the doctor impose a substantial burden on patients and those who care for them.^{8,9}

Therefore, it was sought to determine whether an oral medication that avoids these blinding complications, reduces treatment burden, and lowers health system costs, was effective at improving vision in DME. Lamivudine (also known as 3TC) is a nucleoside analog used to treat retroviral infections. In addition to blocking viral replication, it inhibits the reverse transcription of mobile genetic elements known as short interspersed nuclear elements¹⁰-12 and activation of an innate immune protein complex termed the inflammasome,¹³⁻¹⁵ both of which are implicated in diabetes.^{13,16,17}

Diabetic retinopathy affects the entire retinal neurovascular unit, inducing both neuronal dysfunction and vascular leakage.¹⁸ Aflibercept, bevacizumab, and ranibizumab target the latter, whereas lamivudine protects neuronal function in animal models of retinal dysmetabolism¹⁹ and retina hypoxia²⁰ that characterize diabetic retinopathy. Of note, treatment with a cocktail of reverse transcriptase inhibitors including lamivudine reduced various inflammatory markers in patients with Aicardi-Goutières syndrome.²¹ It was explored whether such beneficial effects of lamivudine might subserve an alternate mechanism of therapeutic efficacy in DME.

To provide efficacy and safety data, a randomized control trial was conducted of oral lamivudine versus oral placebo, with delayed adjunctive intravitreous bevacizumab, for the treatment of DME involving the center of the macula and causing substantial vision impairment. We also compared the results of this trial to those of DRCR.net Protocol T study by using synthetic controls, an approach used by both the United States Food and Drug Administration (FDA) and the European Medicines Agency (EMA) for drug approval.²²

### Methods

### Study Conduct and Oversight

A single center randomized clinical trial was conducted at a tertiary center in São Paulo, Brazil in accordance with the provisions of the Declaration of Helsinki and Good Clinical Practice guidelines. The study was approved by the local institutional review board and study participants provided written informed consent. This study was registered in the Brazilian Registry of Clinical Trials (ReBEC) under the number RBR-87b6r5s. Latinofarma (Brazil) provided the lamivudine and placebo at no cost and also provided funding for bevacizumab which was repackaged in single-used vials by Citopharma (Brazil).

### Participants

Study participants were 18 years of age or older, diagnosed with type 2 diabetes, had a best-corrected visual-acuity letter score (VALS) worse than 69 letters (range, 0 to 100, with higher scores indicating better visual acuity) and center-involved macular edema (CI-DME) measured by spectral-domain optical coherence tomography (OCT) with retinal central subfield thickness of 325 μm or greater. Participants were excluded if they had high-risk proliferative diabetic retinopathy, received any intravitreous injection or laser photocoagulation within the previous 6 months, positive serology for human immunodeficiency virus or hepatitis B virus, or an estimated glomerular rate filtration below 40 mL/min/1.73 m² determined with the use of the Cockcroft-Gault equation. Other eligibility criteria are listed in Table 1.

TABLE 1
Eligibility criteria
Participants

| | |
|---|---|
| Inclusion | Age ≥18 years. |
| | Diagnosis of type 1 or type 2 diabetes. |
| Exclusion | Hemoglobin A1c ≥12%. |
| | Positive serology for HIV or HBV. |
| | Hepatic failure. |
| | Chronic renal disease with an estimated glomerular rate filtration below |
| | 40 mL/min/1.73 m² determined with the use of the Cockcroft-Gault |
| | equation. |
| | Any history of lactic acidosis. |
| | For women of child-bearing potential: pregnant or lactating. |

Study eye

| | |
|---|---|
| Inclusion | Best corrected ETDRS visual acuity letter score ≤69 (worse than 20/50). |
| | Retinal thickening on clinical exam due to DME involving the center of |
| | the macula |
| | Central subfield thickness on SD-OCT ≥325 microns. |
| Exclusion | High-risk proliferative diabetic retinopathy. |
| | History of panretinal photocoagulation within six months prior to |
| | randomization. |
| | History of any treatment for DME in the past six months (such as |
| | intravitreous anti-VEGF, focal or grid macular photocoagulation. |
| | intravitreous or peribulbar corticosteroids). |
| | An ocular condition (other than diabetes) that might affect macular |
| | edema or alter visual acuity during study (e.g. vein occlusion, uveitis, |
| | neovascular glaucoma, etc.). |
| | An ocular condition is present such that, in the opinion of the |
| | investigator, visual acuity loss would not improve from resolution of |
| | macular edema (e.g. foveal atrophy, dense subfoveal hard exudates, or |
| | non-retinal condition). |

HIV = human immunodeficiency virus, HBV = hepatitis B virus, ETDRS = Early Treatmentn Diabetic Retinopathy Study. DME = diabetic macular edema, SD-OCT = spectral-domain optic coherence tomography, VEGF = vascular endothelial growth factor.

### Randomization

Each participant was randomly assigned to be administered oral lamivudine (at a dose of 150 mg twice daily) or oral placebo (twice daily). Randomization was performed at the sealed envelope website (https://sealedenvelope.com), in permuted blocks and with stratification according to visual acuity in the study eye. One physician who was not involved with patient assessment was responsible for the randomization and vial delivery to the patients. The study participants, assessing physicians, and OCT technicians were unaware of the treatment group assignment at all visits.

### Intervention

Lamivudine was administered as an oral solution of 10 mg/ml. Placebo vials were provided by the same pharmaceutical company responsible for lamivudine production using the same vehicle. The vials were concealed and delivered to the study center. Participants took 150 mg of oral lamivudine or placebo from enrollment for 8 weeks. At the 4-week visit, participants were administered an intravitreous injection of bevacizumab (1.25 mg in an injection volume of 0.05 ml) in eyes with CI-DME if the best-corrected visual-acuity letter score (VALS) was less than 69 and the retinal central subfield thickness was 325 μm or more. Injection of bevacizumab was performed by the same physician following topical anesthesia, installation of a sterile lid speculum, and application of povidone-iodine drops. No antibiotic was administered after the procedure.

### Outcome Measurement

Visual acuity assessment was performed by two certified assessing physicians. The same participant could be assessed by a different physician on each visit. The exam was performed using a back-illuminated 4-meter Early Treatment Diabetic Retinopathy Study (ETDRS) chart. Different ETDRS charts were used to refine the refraction and to assess the right and left eye at each visit. If fewer than 20 letters were correctly identified at 4 meters, testing was also performed at 1 meter adding+0.75 sphere. The refraction was performed without pupil dilation, in the same exam room with a standardized background illumination.

Treatment adherence was facilitated by a weekly reminder to the study participants via cell phone message. At each visit, the vials were examined to verify treatment adherence.

If the participant developed high-risk proliferative diabetic retinopathy or had to undergo any ocular surgery during the study period, they would be excluded. Patients were monitored for adverse events by the assessing physician, and new laboratory tests could be performed at the physician's discretion.

### Statistical Assessment

The principal analysis compared lamivudine to placebo. Change in mean VALS from baseline was the primary outcome of interest. We sought to detect a difference of 6 letters in the VALS between groups. This effect size was chosen for power analyses because this change corresponds to a difference of more than 1 line on the ETDRS or Snellen eye charts, the minimal increase in visual acuity judged to be clinically meaningful. By estimating the standard deviation to be 6 letters, we calculated that 16 eyes in each group would provide the trial with 80% power (at a 5% two-sided alpha level) to detect this difference.

To analyze the change in VALS from baseline, a mixed-effects model was employed. Random effects were included in the model to account for subject-specific variability. Baseline visual acuity was included as a covariate to assess the impact of lamivudine while keeping the baseline acuity fixed, as it is a robust predictor of future changes in VALS.^{4,23} Other fixed effect predictors in the model included treatment group (lamivudine vs. placebo), baseline central subfield thickness, time point (week 8 vs. week 4), eye (left vs. right), age, sex (male vs. female), race (Asian vs. White and Black vs. White), and duration of type 2 diabetes. The model's estimates, confidence intervals (CI), and P values were reported for each predictor. Additionally, model-based contrasts were estimated to compare the change in VALS from baseline between lamivudine and placebo at weeks 4 and 8.

The second analysis included participants from the lamivudine versus placebo trial, along with a synthetic comparator created using data from the DRCR.net Protocol T study. The source of the data is DRCR.net (2018) A Comparative Effectiveness Study of Intravitreal Aflibercept, Bevacizumab and Ranibizumab for Diabetic Macular Edema, retrieved from https://public.jaeb.org/drcrnet/stdy/download/206. The analyses, content, and conclusions presented herein are solely the responsibility of the authors and have not been reviewed or approved by DRCR.net.

Inclusion criteria for participants from the DRCR.net Protocol T study were based on their similarity to the lamivudine versus placebo trial design. This involved selecting only patients with type 2 diabetes, study eyes with a baseline VALS<69, and complete follow-up visual acuity measurements at weeks 4 and 8.

Propensity score methods were used to select DRCR.net Protocol T study participants similar to lamivudine-treated patients. Inverse probability treatment weights (IPTW) and propensity score matching were used. To estimate the IPTW weights, a multinomial logistic regression was used to estimate propensity scores using important predictors including baseline visual acuity and baseline central subfield thickness along with age, gender, and duration of diabetes. Weights were calculated as the reciprocal of the estimated propensity score of the actual treatment received. Weights were then truncated at the 99th percentile. For the propensity score matched analysis, lamivudine-treated patients were matched to each DRCR.net Protocol T treatment arm (aflibercept, bevacizumab, ranibizumab) and placebo group, separately. Due to the limited sample size for matching within each group, propensity score models included the most important predictors of acuity change including baseline visual acuity, age and duration of diabetes.

For the IPTW analysis, a mixed-effects weighted model was fit with subject-specific random effects. The fixed effect predictors in the model included treatment group (lamivudine, placebo, aflibercept, bevacizumab, ranibizumab), time, baseline visual acuity, baseline central subfield thickness, eye (left vs. right), age, gender (male vs. female), race (Asian vs. White and Black vs. White), and duration of type 2 diabetes. The model's estimates, confidence intervals (CI), and P values were reported for each predictor. Additionally, model-based contrasts were conducted to compare the change in VALS from baseline between lamivudine and the Protocol T treatments.

For each propensity score matched group (e.g., lamivudine-bevacizumab, lamivudine-aflibercept), a mixed effect model was fit estimating the change in visual acuity from baseline. Subject specific random effect was included along with fixed effect predictors: treatment group, baseline VALS, central subfield thickness, eye (left vs right), age, sex, race and type 2 diabetes duration.

Secondary outcomes included the change in central subfield thickness, the percentage of eyes with an improvement in VALS of 15 or more, the correlation between improvements in visual acuity and central subfield thickness, and systemic and ocular adverse events. Change in central subfield thickness was estimated using mixed-effects models, similar to the VALS analysis. To analyze a VALS improvement of at least 15 letters, logistic regression using generalized estimating equations (GEEs) was specified with an exchangeable correlation matrix. Model-based contrasts were estimated to make pairwise treatment comparisons.

### Initial Clinical Evaluation and Follow-Up

Study participants had a complete eye examination in the Retina sector of the Department of Ophthalmology at Escola Paulista de Medicina-UNIFESP. The exams took place at the beginning of the treatment and after 4 weeks of using the medication. At the 4-week return, all patients were administered an intravitreous injection of Avastin, one of the standard anti-VEGF treatments for DME. Four weeks after the injection (8 weeks from the start of the study) participants underwent another eye examination. Visits within 7 days of these time points were assigned to those time points.

### Eye Exam

Visual acuity: Visual acuity was performed by a masked researcher using a standardized ETDRS table in all consultations. Refraction exam was performed for each patient.

Anterior and posterior biomicroscopy: Performed at every visit, the anterior segment examination included evaluation of the eyelids, conjunctiva, cornea, anterior chamber and iris, lens and intraocular pressure measurement. The follow-up examinations were performed after using 1% tropicamide eye drops and 10% phenylephrine eye drops to induce mydriasis. Optic nerve, central and peripheral retina were examined.

### Spectral Domain-Optical Coherence Tomography

In all consultations, patients underwent tomography with the Spectralis device (Heidelberg) under mydriasis. The acquisition protocol was the 6×6 mm region centered on the fovea. Data analysis from SD-OCT was restricted to the central field (1 mm in diameter) Intravitreous Injection of Anti-VEGF

Anti-VEGF treatment was performed with intravitreous injections of 1.25 mg bevacizumab (Avastin) in 0.05 mL following medicated mydriasis 30 minutes before the procedure and topical anesthesia with 0.5% proxymetacaine hydrochloride and 5% povidone iodine eye drops, using a fenestrated sterile field. The injection was performed 3-3.5 mm from the limbus, with a needle aimed at the center of the eye. After application, no antibiotic eye drops or eye protectors were used.

### Mice

All experiments involving animals were approved by the University of Virginia Institutional Animal Care and Use Committee (IACUC) and adhered to the Association for Research in Vision and Ophthalmology (ARVO) Statement for the Use of Animals in Ophthalmic and Vision Research. Animal subjects were male and female mice aged 6 to 10 weeks. Wild type C₅₇BL/6J mice were obtained from the Jackson Laboratory (Bar Harbor, ME, USA). To anesthetize mice for study procedures, ketamine hydrochloride (100 mg/kg; Ft. Dodge Animal Health, Overland Park, KS, USA) and xylazine (10 mg/kg; Phoenix Scientific, St. Joseph, MO, USA) were delivered by intraperitoneal (IP) injection. Pupils were dilated with topical 1% tropicamide and 2.5% phenylephrine (Alcon Laboratories, Ft. Worth, TX, USA).

### Rhegmatogenous Retinal Detachment Model

RRD model was established by subretinal injection (SRI) of 3 μl 1% carboxymethyl cellulose (CMC; Refresh Liquigel, Allergan, Irvine, CA, USA).

### NRTI and Kamuvudine-9 Treatment

NRTIs and K-9 were delivered via intraperitoneal (IP) injection in equimolar amounts. Lamivudine (3TC, 50 mg/kg, MW=229.26 g/mol) or azidothymidine (AZT, 60 mg/kg, MW=267.24 g/mol) was injected twice daily (SelleckChem, Houston, TX, USA). Kamuvudine K-9 (3-methyl-3TC, MW=273 g/mol) was administered either at a low dose (K-9L; 90 mg/kg daily) or a high dose (K-9H; 60 mg/kg twice daily, equimolar to NRTI doses). IP injection of phosphate buffered saline (PBS) was used as control.

### Immunoblotting

Retinal tissue was extracted and lysed by sonication in radioimmunoprecipitation assay (RIPA) buffer (MilliporeSigma, Darmstadt, Germany). The Pierce BCA Protein Assay Kit (ThermoFisher Scientific, Waltham, MA, USA) was used to determine protein concentrations of interest. Samples were prepared that contained equal concentrations of total protein (10-100 μg). Protein samples were resolved by SDS-PAGE and transferred onto Immobilon-FL PVDF membranes (Millipore, Billerica, MA, USA). Membranes were blocked in Odyssey Blocking Buffer (PBS) for 1 hour at room temperature, and then incubated overnight with primary antibody at 4° C. Across study experiments, the following antibodies were used: anti-mouse caspase-1 (1:1000; AG-20B-0042; AdipoGen Life Sciences, San Diego, CA, USA), and anti-mouse GAPDH (1:1000; 2118; Cell Signaling, Danvers, MA, USA). To visualize immunoreactive bands, species-specific secondary antibodies with conjugated IRDye were applied (1:2000; LI-COR Biosciences, Lincoln, NE, USA). Immunoblot images were captured using the Odyssey Infrared Imaging System (LI-COR Biosciences) or autoradiography film.

### TUNEL Assay

Retinal cross cryosections at a thickness of 10 μm were stained with TUNEL (TdT-mediated dUTP nick-end labeling) using the In Situ Cell Death Detection Kit (Roche Diagnostics, Indianapolis, IN, USA) according to the manufacturer's′ instructions. The sections were wet-mounted with ProLong anti-fade reagent with DAPI (P36935, ThermoFisher Scientific, Waltham, MA, USA) and visualized using an A1R laser confocal microscope (Nikon, Tokyo, Japan). Fourteen representative sections, including the optic nerve, were used per treatment group. To calculate the number of TUNEL+ cells in the outer nuclear layer (ONL), the retina analysis toolkit of ImageJ (2.1.0; NIH, Bethesda, MD, USA) was used.

### Statistical Analyses

Changes in BCVA or OCT CST in patients were analyzed using the Mann Whitney U test. The percentages of patient eyes achieving 10 or 15 BCVA letter improvements were analyzed using Chi Square Test or, in the case of zero cell values, using Fisher's exact test. Outcomes in the mouse studies were analyzed using a Student's two-tailed t-test. P values<0.05 were considered statistically significant. Results were reported as the mean±standard error of the mean (SEM).

### Results

### Clinical Trial

### Study Participants

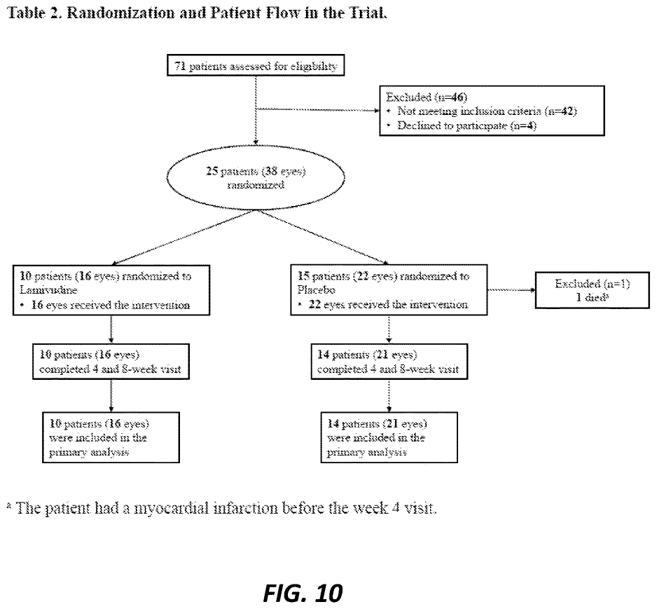

24 participants from a single, tertiary center who completed the 8-week duration of the trial, were randomly assigned to receive oral lamivudine (10 participants; 16 eyes) or placebo (14 participants; 21 eyes) (Table 2 as shown in FIG. 10). The mean age of participants was 62.7±5.9 years; 55% were female; 50% were black, 42% were white, and 8% were Asian. All participants had type 2 diabetes with a mean duration of disease of 13.7±7.0 years and with average glycated hemoglobin of 8.2±1.5%. The baseline VALS was 51.8±13.0 letters (Snellen equivalent, approximately 20/100) and the mean central subfield thickness was 558±190 μm. Baseline characteristics of the groups are summarized in Table 3.

TABLE 3
Characteristics of the Patients at Baseline.

| | | Lamivudine | | Placebo | |
|---|---|---|---|---|---|
| Characteristic | | (N = 16 eyes of 10 subjects) | | (N = 21 eyes of 14 subjects) | |
| Age (years) - Mean (SD) | | 64.1 | (6.7) | 61.64 | (5.3) |
| Sax - N (%) | Female | 6 | (60%) | 7 | (50%) |
| | Male | 4 | (40%) | 7 | (50%) |
| Race/Ethnicity - N (%) | Asian | 0 | (0%) | 2 | (14.3%) |
| | Black | 7 | (70%) | 5 | (35.7%) |
| | White | 3 | (30%) | 7 | (50%) |
| Smoking status - N (%) | Current | 2 | (20%) | 6 | (42.9%) |
| | Never | 5 | (50%) | 6 | (42.9%) |
| | Prior | 3 | (30%) | 2 | (14.3%) |

| | | | | |
|---|---|---|---|---|
| Body mass index - Mean (SD) | 24.6 | (3.4) | 28.5 | (5.0) |
| Type 2 Diabetes - N (%) | 10 | (100%) | 14 | (100%) |
| Duration of Diabetes (years) - Mean (SD) | 14.05 | (7.23) | 13.5 | (7.18) |
| Systolic blood pressure (mm Hg) - Mean (SD) | 145.0 | (20.7) | 132.1 | (13.7) |
| Diastolic blood pressure (mm Hg) - Mean (SD) | 84.0 | (11.7) | 84.3 | (10.2) |
| Mean arterial pressure - Mean (SD) | 104.3 | (13.7) | 100.2 | (10.5) |
| Hemoglobin A1c (%) - Mean (SD) | 8.00 | (1.68) | 8.29 | (1.39) |
| Glomerular filtration rate - Mean (SD) | 62.3 | (14.5) | 79.2 | (25.9) |
| Prior anti-VEGF injection - N (%) | 1 | (10%) | 0 | (0%) |
| Prior panretinal photocoagulation - N (%) | 2 | (20%) | 4 | (28.6%) |
| Prior Ocular hypertension - N (%) | 1 | (10%) | 0 | (0%) |
| Prior Glaucoma - N (%) | 1 | (10%) | 0 | (0%) |
| Prior Chronic kidney disease, Stage 3a - N (%) | 5 | (50%) | 4 | (28.6%) |
| Prior Chronic kidney disease, Stage 2 - N (%) | 4 | (40%) | 4 | (28.6%) |
| Prior Hypertension - N (%) | 5 | (50%) | 10 | (71.4%) |
| Prior Anemis - N (%) | 1 | (10%) | 0 | (0%) |
| Prior Hypercholesterolemia | 1 | (10%) | 4 | (28.6%) |
| Prior Stroke - N (%) | 0 | (0%) | 1 | (7.1%) |
| Prior Myasthenia gravis - N (%) | 0 | (0%) | 1 | (7.1%) |
| Prior Arrhythmia - N (%) | 0 | (0%) | 1 | (7.1%) |
| Prior Depression - N (%) | 0 | (0%) | 1 | (7.1%) |
| Prior Hypothyroidism - N (%) | 1 | (10%) | 1 | (7.1%) |
| Prior Renal transplant - N (%) | 0 | (0%) | 1 | (7.1%) |
| Prior Coronary artery disease - N (%) | 0 | (0%) | 1 | (7.1%) |
| Prior Dilated cardiomyopathy - N (%) | 0 | (0%) | 1 | (7.1%) |

### Effect of Treatment on Visual Acuity

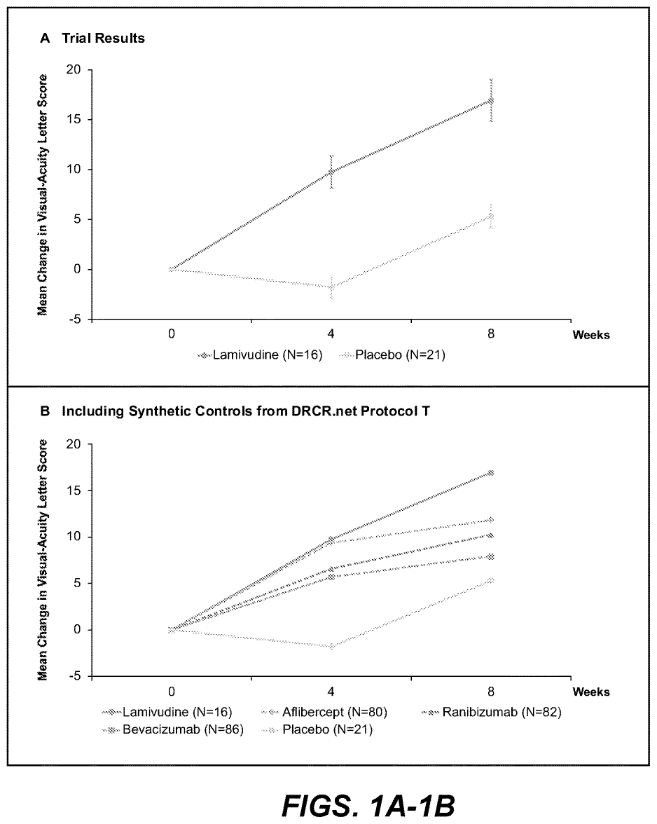

At week 4, before the administration of intravitreous bevacizumab, the mean change in VALS was an increase of 9.8 with lamivudine and a decrease of 1.8 with placebo group (P<0.001). At week 8, following an injection of intravitreous bevacizumab at week 4, the mean improvement in VALS was 16.9 with lamivudine and 5.3 with placebo (P<0.001) (FIG. 1A and Table 4 and 5).

TABLE 4
Mixed model: Change in visual acuity from baseline.

| | Change in Visual Acuity | | |
|---|---|---|---|
| | Estimates | CI | P value |
| Predictors | | | |
| (Intercept) | −4.4 | −37.87-29.07 | .793 |
| Treatment (Lamivudine vs. | 11.86 | 7.71-16.00 | <.001 |
| Placebo) | | | |
| Week 8 vs. week 4 | 7.14 | 4.36-9.91 | <.001 |
| Baseline visual-acuity letter score | −0.16 | −0.30-−0.01 | .033 |
| Baseline central subfield thickness/ | −0.79 | −1.79-0.22 | .122 |
| 100 μm | | | |
| Eye (left vs. right) | 1.72 | −1.24-4.68 | .25 |
| Age (years) | 0.25 | −0.12-0.62 | .187 |
| Gender (Male vs Female) | 3.07 | −0.59-6.73 | .099 |
| Race (Asian vs. White) | 4.43 | −2.73-11.59 | .221 |
| Race (Black vs. White) | −0.25 | −3.84-3.35 | .891 |
| Systolic blood pressure | −0.06 | −0.16-0.03 | .19 |
| Glomerular filtration rate | 0.04 | −0.05-0.13 | .364 |
| Duration of Diabetes (years) | 0.17 | −0.07-0.41 | .173 |
| Random Effects | | | |
| σ² | 35.53 | | |
| τ_{00 ptid} | 0 | | |
| N_{ptid} | 24 | | |
| Observations | 74 | | |
| Marginal R²/Conditional R² | 0.601/NA | | |

TABLE 5
Model-based contrasts: change in visual acuity.

| Contrast | Estimate | 95% CI | P value |
|---|---|---|---|
| Change from baseline to week 4: | 11.86 | 7.8-15.9 | <.001 |
| Lamivudine - Placebo | | | |
| Change from baseline to week 8: | 18.99 | 14.1-23.9 | <.001 |
| Lamivudine - Placebo | | | |

*95% CI is wider than in eTable 3 because simultaneous intervals are shown. P values are uncorrected.

To create a synthetic control, participants were selected from the DRCR.net Protocol T similar to participants in the lamivudine group. A total of 80, 86, and 82 participants were included in the aflibercept, bevacizumab, and ranibizumab groups, respectively. The baseline characteristics of synthetic controls and the lamivudine and placebo groups, before and after IPTW, are summarized in Table 6 and 7. The differences in baseline VALS between the lamivudine group and the treatment groups from the DRCR.net Protocol T study were reduced following IPTW (Table 6 and 7).

TABLE 6
Original baseline patient characteristics including synthetic controls.

| Characteristic | | Placebo | Lamivudine | Bevacizumab | Aflibercept | Ranibizumab |
|---|---|---|---|---|---|---|
| Subjects (N) | | 14 | 10 | 86 | 80 | 82 |
| Eyes (N) | | 21 | 16 | 86 | 80 | 82 |
| Age (years) - Mean (SD) | | 61.6 (5.3) | 64.1 (6.7) | 61.2 (9.0) | 59.5 (10.4) | 60.6 (9.7) |
| Sex - N (%) | Female | 7 (50%) | 6 (60%) | 38 (44.2%) | 41 (51.25%) | 39 (47.6%) |
| | Male | 7 (50%) | 4 (40%) | 48 (55.8%) | 39 (48.75%) | 43 (52.4%) |
| Race - N (%) | Asian | 2 (14.3%) | 0 (0%) | 1 (1.2%) | 2 (2.5%) | 2 (2.4%) |
| | Black | 5 (35.7%) | 7 (70%) | 14 (16.3%) | 10 (12.5%) | 10 (12.2%) |
| | Other | 0 (0%) | 0 (0%) | 4 (4.7%) | 6 (7.5%) | 3 (3.7%) |
| | White | 7 (50%) | 3 (30%) | 67 (77.9%) | 62 (77.5%) | 67 (81.7%) |

| | | | | | |
|---|---|---|---|---|---|
| Type 2 Diabetes - N (%) | 14 (100%) | 10 (100%) | 86 (100%) | 80 (100%) | 82 (100%) |
| Duration of Diabetes | 13.5 (7.2) | 14.1 (7.2) | 17.3 (9.9) | 13.8 (10.0) | 18.0 (10.6) |
| (years) - Mean (SD) | | | | | |
| Baseline visual-acuity | 51.8 (13.0) | 44.3 (13.7) | 57.1 (10. [symbol] ) | 54.7 (11.7) | 56.5 (10.0) |
| letter score* - Mean (SD) | | | | | |
| Baseline central subfield | 570.81 (190.23) | 541.12 (195.71) | 503.1 (144.2) | 513.78 (157.33) | 478.99 (130.25) |
| thickness (μm)* - Mean (SD) | | | | | |

[symbol]

indicates data missing or illegible when filed

TABLE 7
Inverse probability of treatment weighted baseline patient characteristics including synthetic controls.

| Characteristic | | Placebo | Lamivudine | Bevacizumab | Aflibercept | Ranibizumab |
|---|---|---|---|---|---|---|
| Age (years) - Mean | | 60.5 | 64.0 | 60.5 | 62.2 | 60.8 |
| Sex - (%) | Female | 43.4% | 61.4% | 47.9% | 45.4% | 49.3% |
| | Male | 56.6% | 38.6% | 52.1% | 54.6% | 50.7% |
| Race - (%) | Asian | 3.7% | 0.0% | 2.1% | 2.0% | 2.0% |
| | Black | 21.9% | 38.2% | 17.4% | 20.5% | 18.7% |
| | Other | 0.0% | 0.0% | [symbol] | 4.7% | 4.1% |
| | White | 74.4% | 61.8% | 75.3% | 72.7% | 75.2% |

| | | | | | |
|---|---|---|---|---|---|
| Duration of Diabetes (years) - Mean | 14.2 | 14.9 | 16.4 | 16.9 | 16.3 |
| Baseline visual-acuity | 56.7 | 49.6 | 55.5 | 55.0 | 55.6 |
| letter score* - Mean | | | | | |
| Baseline central subfield | 502.1 | 476.3 | 498.9 | 486.5 | 496.1 |
| thickness (μm)* - Mean | | | | | |

[symbol]

indicates data missing or illegible when filed

Using IPTW in a mixed-effects model, which corrected for baseline VALS, the mean improvement in VALS, was 12.2 greater with lamivudine (95% CI, 6.5 to 17.8, P<0.001), 8.4 greater with aflibercept (95% CI, 4.5 to 12.4, P<0.001), 5.3 greater with bevacizumab (95% CI, 1.4 to 9.3, P=0.009), and 7.1 greater with ranibizumab (95% CI, 3.1 to 11.11, P<0.001), compared to the placebo group as the reference (FIG. 1B and Table 8).

TABLE 8
Mixed effect model: Change from baseline visual acuity.

| | Original | | | IPTW | | |
|---|---|---|---|---|---|---|
| Predictors | Estimates | CI | P value | Estimates | CI | P value |
| (Intercept) | 18.71 | 7.76-29.65 | .001 | 14.94 | 4.33-25.55 | .006 |
| Treatment (ref: Placebo) | | | | | | |
| Lamivudine | 11.19 | 5.07-17.31 | <.001 | 12.15 | 6.49-17.82 | <.001 |
| Bevacizumab | 5.36 | 1.04-9.67 | .015 | 5.32 | 1.36-9.29 | .009 |
| Aflibercept | 8.48 | 4.15-12.80 | <.001 | 8.42 | 4.45-12.40 | <.001 |
| Ranibizumab | 7.04 | 2.69-11.38 | .002 | 7.12 | 3.13-11.10 | <.001 |
| Week | 0.84 | 0.65-1.02 | <.001 | 1.15 | 0.96-1.34 | <.001 |
| Baseline visual-acuity letter score | −0.19 | −0.27-−0.11 | <.001 | −0.17 | −0.24-−0.10 | <.001 |
| Baseline central subfield thickness/ | −0.14 | −0.73-0.45 | .635 | 0.03 | −0.52-0.59 | .907 |
| 100 μm | | | | | | |
| Eye (left vs. right) | 1.34 | −0.20-2.89 | .088 | 1.17 | −0.13-2.46 | .077 |
| Age (years) | −0.15 | −0.25-−0.05 | .004 | −0.15 | −0.25-−0.05 | .004 |
| Gender (Male vs. Female) | 0.58 | −1.26-2.41 | .538 | 0.5 | −1.31-2.32 | .587 |
| Race (ref: Asian) | | | | | | |
| Black | −2.96 | −9.11-3.19 | .344 | −2.98 | −9.05-3.09 | .336 |
| Other | −1.2 | −8.28-5.88 | .739 | −1.01 | −8.09-6.07 | .779 |
| White | −1.44 | −7.26-4.39 | .628 | −1.6 | −7.38-4.18 | .587 |
| Duration of Diabetes (years) | −0.06 | −0.16-0.03 | .206 | −0.06 | −0.15-0.04 | .254 |

Random Effects

| | | |
|---|---|---|
| σ² | 20.86 | 6.24 |
| [symbol] | 46.37 [symbol] | 40.64 [symbol] |
| ICC | 0.69 | 0.87 |
| N | 272 [symbol] | 272 [symbol] |
| Observations | 570 | 570 |
| Marginal R²/Conditional R² | 0.205/0.753 | 0.294/0.906 |

[symbol]

indicates data missing or illegible when filed

The results with lamivudine was then directly compared to each of the DRCR.net Protocol T treatment groups using model-based contrasts. The mean difference in the change in VALS from baseline between lamivudine and aflibercept was 3.7 (P=0.13), lamivudine and bevacizumab was 6.8 (P=0.006), and lamivudine and ranibizumab was 5.0 (P=0.042) (Table 9).

TABLE 9
Model-based contrasts: change in visual acuity.

| Contrast | Estimate | 95% CI | P value |
|---|---|---|---|
| Lamivudine vs. Aflibercept | 3.73 | −1.08-8.55 | .131 |
| Lamivudine vs. Bevacizumab | 6.83 | 2.01-11.65 | .006 |
| Lamivudine vs. Ranibizumab | 5.04 | 0.21-9.87 | .042 |

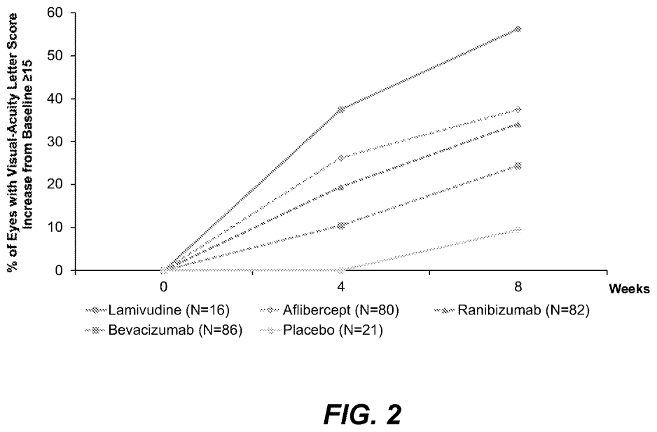

The percentages of eyes with a change in the VALS of 15 or more from baseline are provided in FIG. 2. Among study eyes treated with lamivudine, 56% achieved this level of improvement. In contrast only 10% of placebo eyes did so. Among the included protocol T patients, 24% of eyes treated with bevacizumab, 34% of eyes treated with ranibizumab, and 38% of eyes treated with aflibercept achieved an improvement in VALS of 15 or more from baseline. Using a GEE model with contrast coding, the odds of achieving this level of VALS improvement was estimated accounting for baseline characteristics. Lamivudine had as statistically significantly higher odds of achieving 15 or more VALS improvement when compared to all comparison groups (Table 10 and 11).

TABLE 10
Generalized estimating equation model: Odds of change
in visual acuity letter score from baseline ≥15.

| | Original | | IPTW | |
|---|---|---|---|---|
| Characteristic | Odds Ratio (95% CI) | | Odds Ratio (95% CI) | |
| Intercept | 8.65 | (0.27-274.12) | 10.23 | (0.27-382.19) |
| Treatment (ref = | | | | |
| Placebo) | | | | |
| Lamivudine | 17.19 | (2.73-108.04) | 123.54 | (17.46-873.92) |
| Bevacizumab | 6.15 | (1.17-32.36) | 10.33 | (2.2-48.49) |
| Aflibercept | 11.15 | (2.16-57.65) | 19.39 | (4.12-91.31) |
| Ranibizumab | 11.14 | (2.07-59.87) | 19.96 | (4.29-92.93) |
| Week | 1.25 | (1.15-1.36) | 1.26 | (1.14-1.39) |
| Baseline visual-acuity | 0.94 | (0.91-0.96) | 0.92 | (0.9-0.95) |
| letter score | | | | |
| Baseline central | 0.96 | (0.81-1.14) | 0.95 | (0.77-1.17) |
| subfield thickness/ | | | | |
| 100 μm | | | | |
| Eye (Left vs. Right) | 1.14 | (0.67-1.94) | 0.82 | (0.47-1.45) |
| Age | 0.95 | (0.92-0.98) | 0.95 | (0.92-0.99) |
| Sex (ref = Female) | | | | |
| Male | 1.42 | (0.83-2.43) | 1.31 | (0.71-2.41) |
| Race (ref = Asian) | | | | |
| Black | 0.45 | (0.07-3.09) | 0.23 | (0.03-2.04) |
| Other | 1.29 | (0.19-8.96) | 1.33 | (0.17-10.19) |
| White | 0.8 | (0.14-4.49) | 0.66 | (0.11-4.19) |
| Duration of Diabetes | 0.99 | (0.96-1.01) | 1 | (0.97-1.03) |
| Cluster (Patient ID) | 1 | (1-1.01) | 1 | (1-1.01) |

IPTW, Inverse probability of treatment weighting.

TABLE 11
Model-based contrasts estimated from IPTW model: Odds of
change in visual-acuity letter score from baseline ≥15

| Contrast | Odds Ratio (95% CI) | | P value |
|---|---|---|---|
| Lamivudine vs. Placebo | 123.54 | (17.46-873.9) | <.001 |
| Lamivudine vs. Aflibercept | 6.37 | (1.4-29) | .016 |
| Lamivudine vs. Bevacizumab | 11.95 | (2.59-53.3) | .001 |
| Lamivudine vs. Ranibizumab | 6.19 | (1.33-28.8) | .02 |

IPTW, Inverse probability of treatment weighting.

The results with lamivudine were directly compared to each of the DRCR.net Protocol T treatment groups using propensity score matching and subsequently fitting mixed-effect models, which were corrected for baseline VALS. The baseline characteristics of synthetic controls and the lamivudine and placebo groups are summarized in Table 12. The baseline visual acuity in the lamivudine group was similar to that of the three anti-VEGF drug treatment groups from the DRCR.net Protocol T study (Table 12).

TABLE 12
Propensity score matched baseline patient characteristics including synthetic controls.

| Characteristic | Placebo | Lamivudine | Bevacizumab | Aflibercept | Ranibizumab |
|---|---|---|---|---|---|
| Subjects (N) | 12 | 10 | 16 | 16 | 16 |
| Eyes (N) | 16 | 16 | 16 | 16 | 16 |
| Baseline visual-acuity letter score * - Mean | 47.8 | 44.3 | 44.7 | 44.8 | 45.4 |
| Baseline central subfield thickness (μm) * - | 607.5 | 541.1 | 544.6 | 541.6 | 469.7 |
| Mean | | | | | |
| Eye (Right) - (%) | 50% | 56.25% | 44% | 68.75% | 56.25% |
| Eye (Left) - (%) | 50% | 43.75% | 56% | 31.25% | 43.75% |
| Age (years) - Mean | 62.1 | 64.2 | 58.9 | 59.6 | 59.1 |
| Sex Female - (%) | 56.25% | 56.25% | 37.5% | 62.5% | 75% |
| Male - (%) | 43.75% | 43.75% | 62.5% | 37.5% | 25% |
| Race Asian - (%) | 12.5% | 0% | 0% | 6.25% | 18.75% |
| Black - (%) | 31.25% | 75% | 25% | 18.75% | 6.25% |
| White - (%) | 56.25% | 25% | 68.75% | 6.25% | 75% |
| Other - (%) | 0% | 0% | 6.25% | 68.75% | 0% |
| Type 2 Diabetes - N (%) | 14 (100%) | 14 (100%) | 14 (100%) | 14 (100%) | 14 (100%) |
| Duration of Diabetes (years) - Mean | 14.4 | 14.1 | 18.6 | 13.7 | 17.1 |

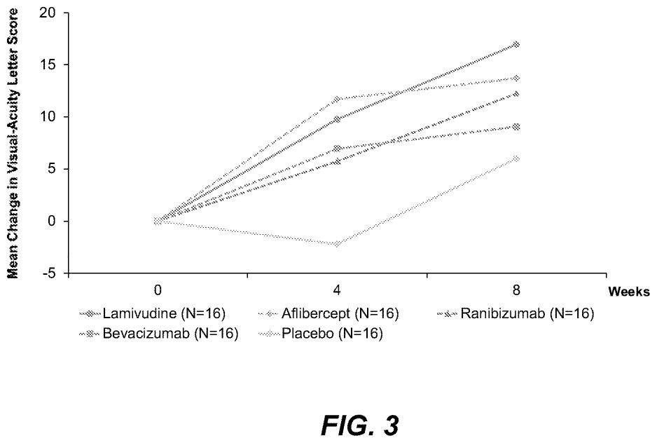

Individual matched cohort results indicate that, on average, there was an improvement in VALS from baseline of 10.9 with lamivudine compared to placebo (P<0.001) (FIG. 3). Comparing lamivudine to the matched synthetic controls, lamivudine had a statistically significantly greater change from baseline in VALS compared to bevacizumab (estimate=9; P=03) and ranibizumab (estimate=7.3, P=0.046). No statistically significant difference was found between lamivudine and aflibercept (estimate=2.3, P=0.5) (Tables 13 to 16).

TABLE 13
Mixed-effect model: Change in visual acuity from baseline between
Lamivudine and Placebo with propensity score matching.

| | Change in visual acuity | | |
|---|---|---|---|
| | Estimates | CI | P value |
| Predictors | | | |
| (Intercept) | −5.75 | −39.25-27.75 | .732 |
| Treatment: Lamivudine vs Placebo | 10.88 | 6.60-15.16 | <.001 |
| Week | 1.92 | 1.13-2.72 | <.001 |
| Baseline visual - acuity letter score | −0.17 | −0.35-0.00 | .052 |
| Baseline central subfield thickness/ | −0.74 | −1.95-0.46 | .222 |
| 100 μm | | | |
| Eye (left ys right) | 2.12 | −1.37-5.62 | .229 |
| Age (years) | 0.14 | −0.26-0.53 | .485 |
| Gender (Male vs. Female) | 2.91 | −1.39-7.20 | .18 |
| Race (Black vs. Asian) | −4.4 | −12.99-4.19 | .308 |
| Race (White vs. Asian) | −4.06 | −11.70-3.57 | .29 |
| Duration of Diabetes (years) | 0.12 | −0.14-0.37 | .363 |
| Random Effects | | | |
| σ² | 40.29 | | |
| τ_{00 PtID} | 0 | | |
| N_{PtID} | 22 | | |
| Observations | 64 | | |
| Marginal R²/Conditional R² | 0.577/NA | | |

TABLE 14
Mixed-effect model: Change in visual acuity from baseline between
Lamivudine and Aflibercept with propensity score matching.

| | Change in Visual Acuity | | |
|---|---|---|---|
| | Estimates | CI | P value |
| Predictors | | | |
| (Intercept) | 23.26 | −8.10-54.62 | .143 |
| Treatment: Lamivudine vs. | 2.33 | −4.49-9.16 | .495 |
| Aflibercept | | | |
| Week | 1.15 | 0.25-2.04 | .013 |
| Baseline visual-acuity letter | −0.15 | −0.36-0.06 | .166 |
| score | | | |
| Baseline central subfield | −0.85 | −2.44-0.74 | .287 |
| thickness/100 μm | | | |
| Eye (left vs. right) | 2.92 | −2.09-7.93 | .247 |
| Age (years) | −0.12 | −0.49-0.25 | .521 |
| Gender (Male vs. Female) | −2.38 | −9.24-4.47 | .488 |
| Race (Black vs. Asian) | 2.04 | −14.42-18.49 | .805 |
| Race (Other vs. Asian) | −6.85 | −28.12-14.43 | .521 |
| Race (White vs. Asian) | 7 | −8.48-22.49 | .368 |
| Duration of Diabetes (years) | −0.28 | −0.63-0.07 | .116 |
| Random Effects | | | |
| σ² | 50.73 | | |
| τ_{00 PtID} | 26.16 | | |
| ICC | 0.34 | | |
| N_{PtID} | 26 | | |
| Observations | 64 | | |
| Marginal R²/Conditional R² | 0.266/0.516 | | |

TABLE 15
Mixed-effect model: Change in visual acuity from baseline between
Lamivudine and Bevacizumab with propensity score matching.

| | Change in Visual Acuity | | |
|---|---|---|---|
| | Estimates | CI | P value |
| Predictors | | | |
| (Intercept) | 2.22 | −33.40-37.84 | .901 |
| Treatment: Lamivudine vs. | 8.95 | 0.94-16.95 | .029 |
| Bevacizumab | | | |
| Week | 1.16 | 0.34-1.99 | .007 |
| Baseline visual-acuity letter | 0.04 | −0.19-0.27 | .71 |
| score | | | |
| Baseline central subfield | −0.23 | −1.87-1.41 | .783 |
| thickness/100 μm | | | |
| Eye (left vs. right) | 1.11 | −3.52-5.75 | .632 |
| Age (years) | −0.12 | −0.65-0.41 | .649 |
| Gender (Male vs. Female) | −2.23 | −8.81-4.35 | .499 |
| Race (Other vs. Black) | 14.61 | −3.32-32.54 | .108 |
| Race (White vs. Black) | 4.65 | −3.46-12.76 | .255 |
| Duration of Diabetes (years) | 0.1 | −0.22-0.43 | .525 |
| Random Effects | | | |
| σ² | 43.28 | | |
| τ_{00 PtID} | 37.9 | | |
| ICC | 0.47 | | |
| N_{PtID} | 26 | | |
| Observations | 64 | | |
| Marginal R²/Conditional R² | 0.212/0.580 | | |

TABLE 16
Mixed-effect model: Change in visual acuity from baseline between
Lamivudine and Ranibizumab with propensity score matching.

| | Change in Visual Acuity | | |
|---|---|---|---|
| | Estimates | CI | P value |
| Predictors | | | |
| (Intercept) | 13.59 | −21.10-48.28 | .435 |
| Treatment: Lamivudine vs. | 7.24 | 0.12-14.37 | .046 |
| Ranibizumab | | | |
| Week | 1.71 | 0.93-2.50 | <.001 |
| Baseline visual-acuity letter | −0.12 | −0.34-0.11 | .303 |
| score | | | |
| Baseline central subfield | −1.39 | −3.43-0.65 | .178 |
| thickness/100 μm | | | |
| Eye (left vs. right) | 3.9 | −0.48-8.29 | .08 |
| Age (years) | −0.09 | −0.48-0.30 | .641 |
| Gender (Male vs. Female) | 2.31 | −3.94-8.56 | .462 |
| Race (Other vs. Black) | 18.61 | 1.18-36.05 | .037 |
| Race (White vs. Black) | 3.24 | −3.54-10.02 | .342 |
| Duration of Diabetes (years) | −0.21 | −0.67-0.25 | .368 |
| Random Effects | | | |
| σ² | 39.13 | | |
| τ_{00 PtID} | 30.27 | | |
| ICC | 0.44 | | |
| N_{PtID} | 26 | | |
| Observations | 64 | | |
| Marginal R²/Conditional R² | 0.381/0.651 | | |

### Effect of Treatment on Retinal Thickening

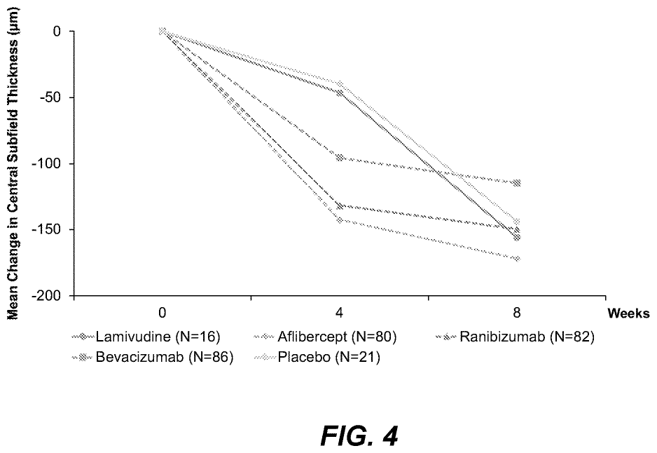

At week 4, there were no statistically significant differences in reduction of central subfield thickness from baseline between lamivudine and placebo (P=0.25) or bevacizumab (P=0.35), whereas there were significant differences between lamivudine and aflibercept (P=0.03) or ranibizumab (P=02) (FIG. 4 and Table 17 and 18). By week 8, following the injection of intravitreous bevacizumab at week 4, no statistically significant differences in the change in central subfield thickness were detected between lamivudine and any of the other treatments. Following IPTW, there were no significant differences in change in central subfield thickness between lamivudine and placebo or bevacizumab at weeks 4 or 8 (Table 19 and 20). At week 4, the difference in mean reduction in central subfield thickness between lamivudine and aflibercept was 55 μm (P=0.07) and between lamivudine and ranibizumab was 65 μm (P=0.03). At week 8, there were no significant differences in change in central subfield thickness between lamivudine and aflibercept or ranibizumab.

TABLE 17
Mixed-effect model: Change in central
subfield thickness from baseline.

| | Change in central subfield thickness | | |
|---|---|---|---|
| | Estimates | CI | P value |
| Predictors | | | |
| (Intercept) | 159.58 | 30.56-288.60 | .015 |
| Treatment (ref = Placebo) | | | |
| Lamivudine | −44.91 | −121.31-31.50 | .249 |
| Bevacizumab | −75.55 | −129.33-−21.77 | .006 |
| Aflibercept | −114.55 | −168.49-−60.61 | <.001 |
| Ranibizumab | −122.12 | −176.28-−67.96 | <.001 |
| Week 8 vs. 4 | −104.1 | −132.83-−75.36 | <.001 |
| Baseline visual-acuity letter | −0.8 | −1.70-0.10 | .082 |
| score | | | |
| Baseline central subfield | −53.59 | −60.35-−46.82 | <.001 |
| thickness/100 μm | | | |
| Eye [Left] | −2.72 | −20.19-14.74 | .76 |
| Age | 2 | 0.79-3.22 | .001 |
| Gender [Male] | 14.42 | −7.29-36.14 | .193 |
| Race [Black] | 32.37 | −40.41-105.15 | .383 |
| Race [Other] | 6.87 | −76.93-90.66 | .872 |
| Race [White] | 5.19 | −63.78-74.15 | .883 |
| Duration of Diabetes | −0.16 | −1.31-0.98 | .779 |
| Treatment [Lamivudine] × | 3.8 | −40.43-48.04 | .866 |
| week [8] | | | |
| Treatment [Bevacizumab] × | 84.87 | 52.82-116.93 | <.001 |
| week [8] | | | |
| Treatment [Aflibercept] × | 74.83 | 42.54-107.12 | <.001 |
| week [8] | | | |
| Treatment [Ranibizumab] × | 85.69 | 53.41-117.96 | <.001 |
| week [8] | | | |
| Random Effects | | | |
| σ² | 2247.12 | | |
| τ_{00 PtID} | 6839.3 | | |
| ICC | 0.75 | | |
| N_{PtID} | 272 | | |
| Observations | 567 | | |
| Marginal R²/Conditional R² | 0.463/0.867 | | |

TABLE 18
Model-based contrasts: change in central subfield thickness.

| Contrast | Week | Estimates | CI | P value |
|---|---|---|---|---|
| Lamivudine vs | 4 | −44.91 | −121.14-31.3 | .248 |
| Placebo | 8 | −41.1 | −117.06-34.9 | .289 |
| Lamivudine vs. | 4 | 69.64 | 5.61-133.7 | .033 |
| Aflibercept | 8 | −1.39 | −65.15-62.4 | .966 |
| Lamivudine vs | 4 | 30.65 | −33.31-94.6 | .348 |
| Bevacizumab | 8 | −50.42 | −114.09-13.2 | .121 |
| Lamivudine vs. | 4 | 77.22 | 13.01-141.4 | .018 |
| Ranibizumab | 8 | −4.66 | −68.65-59.3 | .886 |

TABLE 19
Inverse probability of treatment weighted mixed-effect model:
Change in central subfield thickness from baseline.

| | Change from baseline | | |
|---|---|---|---|
| | Estimates | CI | P value |
| Predictors | | | |
| (Intercept) | 128.82 | 3.18-254.46 | .044 |
| Treatment (ref = Placebo) | | | |
| Lamivudine | −48.84 | −118.15-20.47 | .167 |
| Bevacizumab | −66.35 | −115.60-−17.09 | .008 |
| Aflibercept | −104.33 | −153.63-−55.04 | <.001 |
| Ranibizumab | −113.67 | −163.16-−64.19 | <.001 |
| Week 8 vs 4 | −94.99 | −114.88-−75.10 | <.001 |
| Baseline visual-acuity letter | −0.4 | −1.22-0.43 | .344 |
| score | | | |
| Baseline central subfield | −52.72 | −59.40-−46.04 | <.001 |
| thickness/100 μm | | | |
| Eye [Left] | 6.65 | −9.25-22.54 | .412 |
| Age | 1.94 | 0.75-3.14 | .002 |
| Gender [Male] | 12.28 | −9.15-33.72 | .261 |
| Race [Black] | 23.66 | −48.44-95.76 | .519 |
| Race [Other] | −1.59 | −85.38-82.21 | .97 |
| Race [White] | 0.1 | −68.57-68.76 | .998 |
| Duration of Diabetes | −0.17 | −1.30-0.96 | .771 |
| Treatment [Lamivudine] × | 41.98 | 9.31-74.65 | .012 |
| week [8] | | | |
| Treatment [Bevacizumab] × | 74.82 | 46.75-102.89 | <.001 |
| week [8] | | | |
| Treatment [Aflibercept] × | 68.54 | 40.95-96.14 | <.001 |
| week [8] | | | |
| Treatment [Ranibizumab] × | 75 | 46.88-103.12 | <.001 |
| week [8] | | | |
| Random Effects | | | |
| σ² | 14213.88 | | |
| τ_{00 PtID} | 5686.38 | | |
| ICC | 0.29 | | |
| N_{PtID} | 272 | | |
| Observations | 567 | | |
| Marginal R²/Conditional R² | 0.276/0.483 | | |

TABLE 20
Inverse probability of treatment weighted model-based
contrasts: change in central subfield thickness.

| Contrast | Week | Estimate | CI | P value |
|---|---|---|---|---|
| Lamivudine vs | 4 | −48.84 | −118-20.3 | .166 |
| Placebo | 8 | −6.86 | −75.91-62.2 | .846 |
| Lamivudine vs. | 4 | 55.49 | −3.78-114.8 | .066 |
| Aflibercept | 8 | 28.93 | −30.21-88.1 | .338 |
| Lamivudine vs | 4 | 17.5 | −41.9-76.9 | .564 |
| Bevacizumab | 8 | −15.34 | −74.61-43.9 | .612 |
| Lamivudine vs. | 4 | 64.83 | 5.38-124.3 | .033 |
| Ranibizumab | 8 | 31.81 | −27.55-91.2 | .294 |

Using the propensity score matched model, there were no significant differences in reduction in central subfield thickness between lamivudine and placebo or bevacizumab at weeks 4 or 8 (Table 21). At week 4, the difference in mean reduction in central subfield thickness between lamivudine and aflibercept was 93 μm (P=0.051) and between lamivudine and ranibizumab was 116 μm (P=0.013). At week 8, there were no significant differences mean reduction in central subfield thickness between lamivudine and aflibercept or ranibizumab.

TABLE 21
Propensity score matched model-based contrasts:
change in central subfield thickness.

| Contrast | Week | Estimate | CI | P value |
|---|---|---|---|---|
| Lamivudine vs. | 4 | −47.7 | −130.412-35.012 | .260 |
| Placebo | 8 | −38.1 | −120.224-44.024 | .360 |
| Lamivudine vs. | 4 | 92.6 | −0.304-185.504 | .051 |
| Aflibercept | 8 | 21.2 | −71.508-113.908 | .654 |
| Lamivudine vs. | 4 | 12.6 | −106.568-131.768 | .840 |
| Bevacizumab | 8 | −63.4 | −182.372-55.572 | .300 |
| Lamivudine vs. | 4 | 115.7 | 24.56-206.84 | .013 |
| Ranibizumab | 8 | 37.1 | −53.648-127.848 | .423 |

Estimates arise from propensity score matched data (e.g. Lamivudine-Ranibizumab, Lamivudine-Placebo) and estimated from the models fit from the respective matched dataset.

At weeks 4 and 8, there were no correlations between baseline central subfield thickness and improvement in VALS with lamivudine, placebo, aflibercept, bevacizumab, or ranibizumab (Table 22). There were no correlations between change in central subfield thickness and improvement in VALS with lamivudine or placebo, and either no or weak correlations with aflibercept, bevacizumab, or ranibizumab (Table 23).

TABLE 22
Correlations between baseline central subfield thickness
and change in visual acuity from baseline.

Week 4

| Group | r² | P value |
|---|---|---|
| Placebo | 0.007 | 1.0 |
| Lamivudine | 0.214 | .71 |
| Aflibercept | 0.040 | .71 |
| Bevacizumab | 0.005 | 1.0 |
| Ranibizumab | 0.033 | 1.0 |

Week 8

| Group | r² | P value |
|---|---|---|
| Placebo | 0.084 | 1.0 |
| Lamivudine | 0.033 | 1.0 |
| Aflibercept | 0.033 | .71 |
| Bevacizumab | 0.037 | .71 |
| Ranibizumab | 0.033 | .71 |

P values corrected using Holm-Bonferroni method for multiple hypothesis testing.

TABLE 23
Correlations between change in central subfield thickness
from baseline and change in visual acuity from baseline.

Week 4

| Group | r² | P value |
|---|---|---|
| Placebo | <0.001 | .99 |
| Lamivudine | 0.036 | .99 |
| Aflibercept | 0.096 | .04 |
| Bevacizumab | 0.052 | .20 |
| Ranibizumab | 0.047 | .26 |

Week 8

| Group | r² | P value |
|---|---|---|
| Placebo | 0.162 | .28 |
| Lamivudine | 0.130 | .51 |
| Aflibercept | 0.108 | .03 |
| Bevacizumab | 0.077 | .07 |
| Ranibizumab | 0.099 | .04 |

P values corrected using Holm-Bonferroni method for multiple hypothesis testing.

At week 8, in the lamivudine group, 64% of eyes with an improvement in the VALS of 15 or more had minimal improvement in central subfield thickness (≤50 μm). A similar divergence between marked improvement in visual acuity and improvement in central subfield thickness was observed in the synthetic controls at week 8 of the DRCR.net Protocol T study: 42% of eyes treated with aflibercept, 29% with bevacizumab, and 41% with ranibizumab that had an improvement in the VALS of 15 or more had minimal change in central subfield thickness (≤50 μm).

### Safety

### Ocular Adverse Events

At week 4, there were no ocular adverse events either with lamivudine or placebo. At week 8, the only ocular adverse event reported was subconjunctival hemorrhage after intravitreous injection, occurring in three participants in each group. No one in either treatment group developed high-risk proliferative diabetic retinopathy or underwent any ocular surgery during the study period.

### Systemic Adverse Events

During the period of the study, there were no serious systemic adverse events reported. The systemic adverse events reported were self-limited and lasted no more than 4 days. These included abdominal pain, diaphoresis, diarrhea, fatigue, headache, nausea or vomiting, and pruritus, the incidence of all of which were not significantly different between the two groups (Table 24).

TABLE 24
Adverse events.

| Event | Lamivudine | | Placebo | | P value |
|---|---|---|---|---|---|
| Systemic adverse events | | | | | |
| Subjects, N | 10 | | 14 | | |
| Headache - N (%) | 2 | (20%) | 2 | (14.3%) | 1.0 |
| Nausea or vomiting - N (%) | 2 | (20%) | 2 | (14.3%) | 1.0 |
| Abdominal pain - N (%) | 0 | (0%) | 1 | (7.1%) | 1.0 |
| Diarrhea - N (%) | 1 | (10%) | 2 | (14.3%) | 1.0 |
| Fatigue - N (%) | 1 | (10%) | 1 | (7.1%) | 1.0 |
| Pruritus - N (%) | 2 | (20%) | 0 | (0%) | .16 |
| Disphoresis - N (%) | 1 | (10%) | 0 | (0%) | .42 |
| Alopecia - N (%) | 0 | (0%) | 0 | (0%) | 1.0 |
| Skin rash - N (%) | 0 | (0%) | 0 | (0%) | 1.0 |
| Arthralgia - N (%) | 0 | (0%) | 0 | (0%) | 1.0 |
| Myalgia - N (%) | 0 | (0%) | 0 | (0%) | 1.0 |
| Ocular adverse events | | | | | |
| Eyes, N | 16 | | 21 | | |
| Subconjunctival hemorrhage - | 3 | (18.8%) | 3 | (14.3%) | 1.0 |
| N (%) | | | | | |
| Retinal detachment - N (%) | 0 | (0%) | 0 | (0%) | 1.0 |
| Traumatic cataract - N (%) | 0 | (0%) | 0 | (0%) | 1.0 |
| Vitreous hemorrhage - N (%) | 0 | (0%) | 0 | (0%) | 1.0 |
| Intraocular inflammation* - N (%) | 0 | (0%) | 0 | (0%) | 1.0 |
| Endophthalmitis - N (%) | 0 | (0%) | 0 | (0%) | 1.0 |
| Increase in intraocular | 0 | (0%) | 0 | (0%) | 1.0 |
| pressure ≥10 mm Hg from | | | | | |
| baseline - N (%) | | | | | |

P values by Fisher Exact Test.

### Animal Model of Rhegmatogenous Retinal Detachment

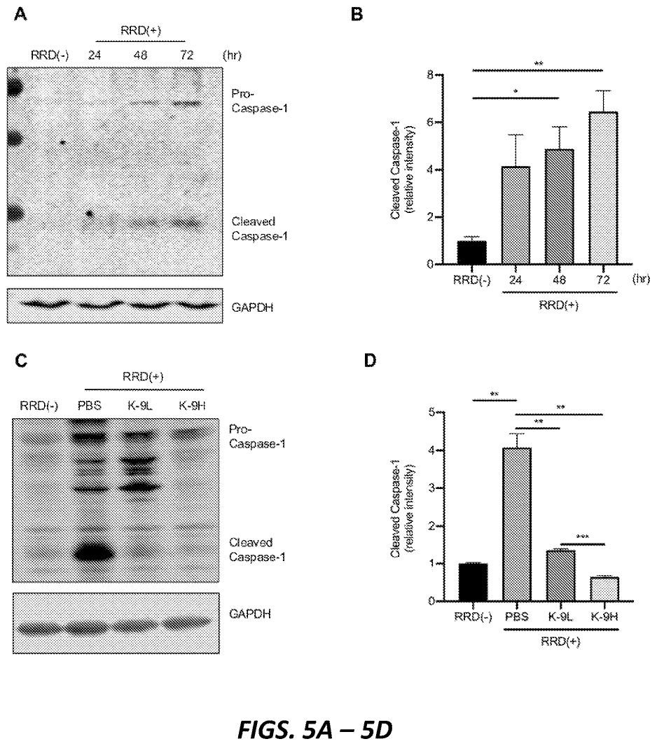

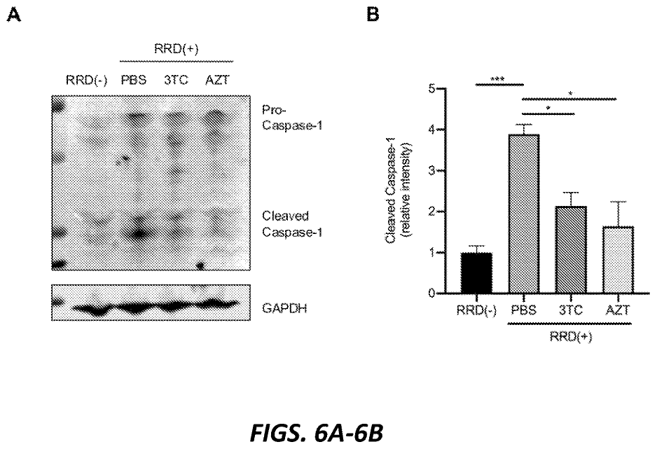

Experimental Rhegmatogenous Retinal Detachment (RRD) induced a progressive increase in the abundance of cleaved caspase-1 in retinal lysates isolated from mice over 24-72 hours (FIGS. 5A, 5B). These data indicate in vivo inflammasome activation following RRD and are consistent with hypoxia-induced inflammasome activation. Three days after inducing RRD, mice treated with systemic K-9 exhibited dose-dependent and complete reduction in retinal cleaved caspase-1, compared with PBS-treated mice (FIGS. 5C, 5D). Three days after inducing RRD, mice treated with either of the NRTIs (3TC or AZT), exhibited a marked reduction in retinal caspase-1 levels, compared with PBS-treated mice (FIGS. 6A, 6B). These results are compatible with the known inflammasome inhibitory activity of K-9 and NRTIs. Of note, equimolar amounts of K-9H exerted a greater degree of suppression of caspase-1 activation than the 3TC (84%+1% versus 52%+8%, P<0.05).

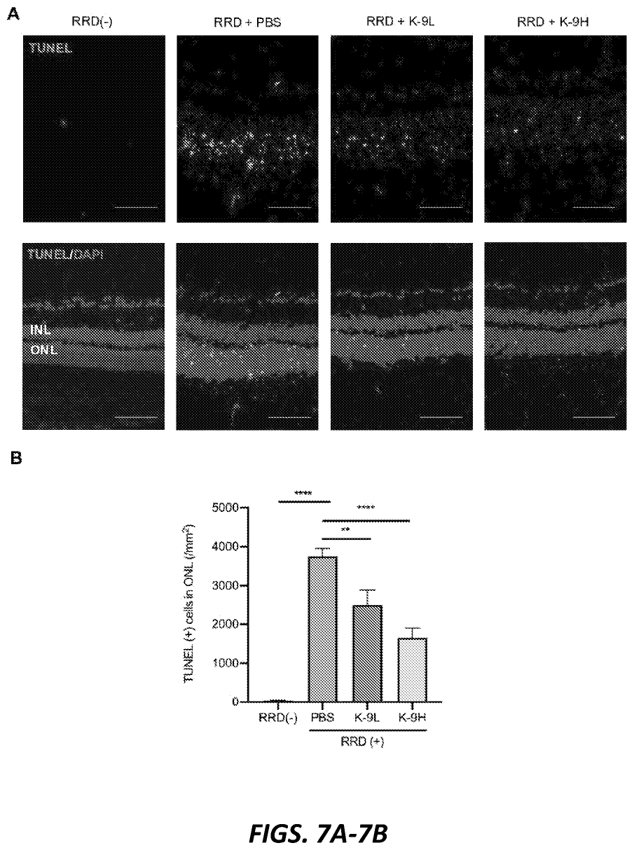

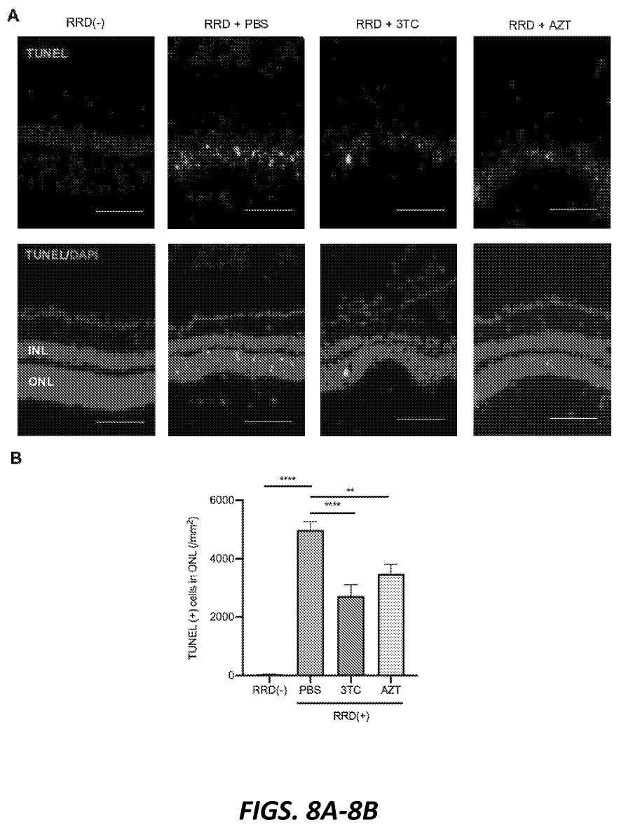

Next, retinal neuroprotection was analyzed by assessing TUNEL staining to measure dead or dying cells. Three days after inducing RRD, a dose-dependent reduction was observed in the number of TUNEL+ cells in the ONL of the retina following K-9 treatment, compared with PBS-treatment (FIG. 7). Similarly, mice treated with systemic 3TC or AZT displayed significantly fewer TUNEL+ retinal cells, compared with PBS-treated mice (FIG. 8). These findings demonstrate that systemic delivery of these three inflammasome inhibitors can protect photoreceptors from damage during periods of RRD. Of note, equimolar amounts of K-9H conferred a greater degree of photoreceptor protection than the 3TC (56%+7% versus 38%+10%, P<0.05).

### 3TC, AZT, K9, and K8 Protect Retinal Function in a Mouse Model of Diabetic Macular Edema.

### Drug Administration

Drugs were delivered via intravitreous (IVt) or intraperitoneal (IP) injections in equimolar amounts. K-8 (0.5 nmol) was administered via IVt in a 0.5 L volume. The same concentration of DMSO in phosphate buffered saline (PBS) was used as control. 3TC (50 mg/kg), AZT (60 mg/kg) and K-9 (60 mg/kg) were administered twice daily via IP administration. IP administration of PBS was used as control.

### ERG

ERG recordings were performed at baseline, as well as on day 20 following IVt administration of 500 ng VEGF on days 0 and 1. NRTI and Kamuvudine administrations were performed twice daily from day −1 to day 20 for IP administration, and day −1 to day 2 for IVt administration. Scotopic ERG was recorded using Phoenix Ganzfeld ERG system (Phoenix Research Labs) after 12 h dark adaptation. Before recording, mice were anesthetized with intraperitoneal injection of Ketamine and Xylazine. Pupils were dilated with 0.5% tropicamide and 2.5% phenylephrine to induce complete mydriasis. During the recording, mice were placed on heating pad (Phoenix Research Labs). Electrical contact was placed on the cornea. The reference needle electrode was placed subdermal between the eyes and the ground electrode was placed on the tail. ERGs were evoked by 504 nm green light stimuli. All measurements were done in dark room with the aim of dim red light. 1.0 log cd s/m² stimulations were chosen to see mixed rod and cone system responses. Averaging was performed with 3 sweeps with 60 sec intervals. The amplitude of a-wave was measured as the difference between the trough of a-wave to baseline and the amplitude of b-wave was measured as the difference between the trough of a-wave to peak of b-wave.

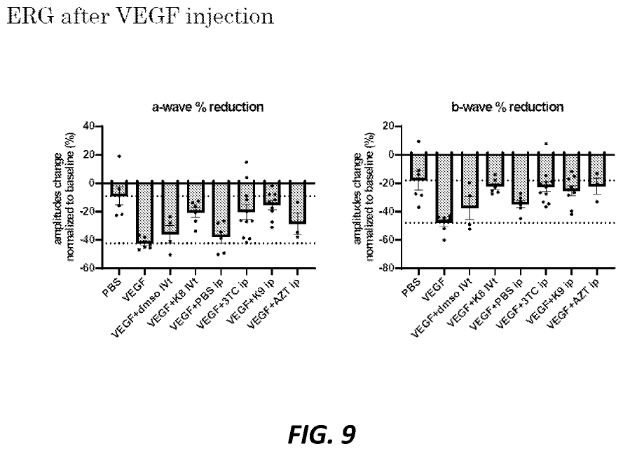

FIG. 9 demonstrates that 3TC, AZT, K9, and K8 protect retinal function in a mouse model of diabetic macular edema.

### Discussion

In this randomized clinical trial of CI-DME associated with low vision (VALS less than 69), oral lamivudine alone was significantly better than oral placebo in improving visual acuity after 4 weeks (+9.8 vs. −1.8 letters for lamivudine and placebo, respectively). After the administration of a single intravitreous bevacizumab injection at week 4, a similar rate of improvement in visual acuity was observed at week 8 (+16.9 vs. +5.3 letters for lamivudine and placebo, respectively). There were higher rates of clinically meaningful vision improvement (15 letters or more, i.e., 3 Snellen lines) with lamivudine (38% and 56% at weeks 4 and 8, respectively) than with placebo (0% and 10%, at weeks 4 and 8, respectively).

Evaluating historical external data using statistical methods to create synthetic controls is a validated approach that has enabled drugs such as alectinib, avelumab, blinatumomab, cerliponase, and palbociclib to receive approval or expanded indications from the FDA and EMA.²² The use of synthetic controls matched for important clinical and demographic variables enabled one to make a meaningful comparison between our trial and DRCR.net Protocol T results using the same time points. Using an IPTW model, visual acuity improvement with lamivudine was significantly greater than with all three anti-VEGF drugs studied in DRCR.net Protocol T. Using a propensity-score-matched model, visual acuity improvement with lamivudine was significantly greater than with bevacizumab and ranibizumab and not significantly different than with aflibercept.

The highest rate of visual acuity improvement with anti-VEGF drugs in the DRCR.net Protocol T study as well as other clinical trials in DME occurs during the first 4 weeks of treatment, with the rate of visual gains diminishing thereafter. Thus, it is notable that visual acuity improved substantially with lamivudine not only in the first 4 weeks but also in the next 4 weeks after intravitreous bevacizumab administration. Further, at week 8, a clinically meaningful advantage (greater improvement in the VALS of at least 15) was observed in more eyes of subjects treated with oral lamivudine and intravitreous bevacizumab than in the synthetic comparator aflibercept-treated (56% vs. 38%), bevacizumab-treated (56% vs. 24%), and ranibizumab-treated eyes (56% vs. 34%). These findings provide an additive effect between lamivudine and anti-VEGF therapy.

All existing approved pharmacotherapies for DME reduce vascular permeability. Therefore, it is notable that visual acuity improved with lamivudine at week 4 without a concomitant improvement in central subfield thickness. This finding suggests that the functional improvement in vision could be a result of the neuroprotective effect of lamivudine observed in preclinical disease models^{19,20} and offers proof of concept that inflammasome inhibition could be therapeutic in DME and possibly other retinal disorders via this novel mechanism of action.

Despite the temporally concurrent improvement in central subfield thickness and visual acuity observed with anti-VEGF drugs in DME, there is a low correlation (r²=11) between change in central subfield thickness and visual acuity improvement at the 2 year time point in the DRCR.net Protocol T study.²⁴ A meta-analysis of DME studies also reported no significant association between change in central subfield thickness and visual acuity improvement.²⁵ Such disconnects between anatomical and functional improvement have been cited by the FDA for not approving central subfield thickness as an endpoint.²⁶

The advent of anti-VEGF drugs was a landmark in the treatment of DME. However, continual intravitreous administration of these medications requiring frequent visits to doctors' offices imposes a substantial burden on patients and health care systems. An oral therapy of a generic medication such as lamivudine could obviate these challenges by reducing medication cost and eliminating intravitreous injection cost, and it would also avoid the potentially blinding complications of intravitreous injections. This therapeutic paradigm can be particularly impactful for underserved populations for whom regular access to eye specialists is a barrier to treatment.

Among adults with type 2 diabetes, the use of lamivudine, an orally administered inflammasome inhibitor, is an effective and relatively safe treatment for CI-DME causing vision impairment.

### BIBLIOGRAPHY

1. Lundeen E A, Burke-Conte Z, Rein D B, et al. Prevalence of Diabetic Retinopathy in the US in 2021. JAMA Ophthalmol. 2023; 141 (8): 747-754. doi:10.1001/jamaophthalmol.2023.2289
2. International Diabetes Federation. IDF Diabetes Atlas. 10th ed. International Diabetes Federation; 2021.
3. Baker C W, Glassman A R, Beaulieu W T, et al. Effect of Initial Management With Aflibercept vs Laser Photocoagulation vs Observation on Vision Loss Among Patients With Diabetic Macular Edema Involving the Center of the Macula and Good Visual Acuity: A Randomized Clinical Trial. JAMA. 2019; 321(19):1880-1894. doi:10.1001/jama.2019.5790
4. Diabetic Retinopathy Clinical Research N, Wells J A, Glassman A R, et al. Aflibercept, bevacizumab, or ranibizumab for diabetic macular edema. N Engl J Med. 2015; 372(13):1193-1203. doi:10.1056/NEJMoa1414264
5. Storey P P, Pancholy M, Wibbelsman T D, et al. Rhegmatogenous Retinal Detachment after Intravitreal Injection of Anti-Vascular Endothelial Growth Factor. Ophthalmology. 2019; 126(10):1424-1431. doi:10.1016/j.ophtha.2019.04.037
6. Mun Y, You S C, Lee D Y, et al. Real-world incidence of endopthalmitis after intravitreal anti-VEGF injections in Korea: findings from the Common Data Model in ophthalmology. Epidemiol Health. 2021; 43:e2021097. doi:10.4178/epih.e2021097
7. Martin D F. Evolution of Intravitreal Therapy for Retinal Diseases—From CMV to CNV: The LXXIV Edward Jackson Memorial Lecture. Am J Ophthalmol. 2018; 191: xli-lviii. doi:10.1016/j.ajo.2017.12.019
8. Shahzad H, Mahmood S, McGee S, et al. Non-adherence and non-persistence to intravitreal anti-vascular endothelial growth factor (anti-VEGF) therapy: a systematic review and meta-analysis. Syst Rev. 2023; 12 (1): 92. doi:10.1186/s13643-023-02261-x
9. Spooner K L, Guinan G, Koller S, Hong T, Chang A A. Burden Of Treatment Among Patients Undergoing Intravitreal Injections For Diabetic Macular Oedema In Australia. Diabetes Metab Syndr Obes. 2019; 12:1913-1921. doi:10.2147/DMSO.S214098
10. Fukuda S, Narendran S, Varshney A, et al. Alu complementary DNA is enriched in atrophic macular degeneration and triggers retinal pigmented epithelium toxicity via cytosolic innate immunity. Sci Adv. 2021; 7 (40): eabj3658. doi:10.1126/sciadv.abj3658
11. Kazazian H H Jr, Moran J V. Mobile DNA in Health and Disease. N Engl J Med. 2017; 377 (4): 361-370. doi:10.1056/NEJMra1510092
12. Fukuda S, Varshney A, Fowler B J, et al. Cytoplasmic synthesis of endogenous Alu complementary DNA via reverse transcription and implications in age-related macular degeneration. Proc Natl Acad Sci USA. 2021; 118 (6):e2022751118. doi:10.1073/pnas.2022751118
13. Ambati J, Magagnoli J, Leung H, et al. Repurposing anti-inflammasome NRTIs for improving insulin sensitivity and reducing type 2 diabetes development. Nat Commun. 2020; 11 (1): 4737. doi:10.1038/s41467-020-18528-z
14. Rosenbaum J T. Eyeing macular degeneration—few inflammatory remarks. N Engl J Med. 2012; 367 (8): 768-770. doi:10.1056/NEJMcibr1204973
15. Fowler B J, Gelfand B D, Kim Y, et al. Nucleoside reverse transcriptase inhibitors possess intrinsic anti-inflammatory activity. Science. 2014; 346 (6212): 1000-1003. doi:10.1126/science. 1261754
16. Masters S L, Dunne A, Subramanian S L, et al. Activation of the NLRP3 inflammasome by islet amyloid polypeptide provides a mechanism for enhanced IL-1β in type 2 diabetes. Nat Immunol. 2010; 11 (10): 897-904. doi:10.1038/ni.1935
17. Yan Y, Salazar T E, Dominguez J M 2nd, et al. Dicer expression exhibits a tissue-specific diurnal pattern that is lost during aging and in diabetes. PLOS One. 2013; 8 (11):e80029. doi:10.1371/journal.pone.0080029
18. Antonetti D A, Klein R, Gardner T W. Diabetic retinopathy. N Engl J Med. 2012; 366 (13): 1227-1239. doi:10.1056/NEJMra1005073
19. Pavlou S, Augustine J, Cunning R, et al. Attenuating Diabetic Vascular and Neuronal Defects by Targeting P2rx7. Int J Mol Sci. 2019; 20 (9): 2101. doi:10.3390/ijms20092101
20. Huang P, Thomas C C, Ambati K, et al. Kamuvudine-9 Protects Retinal Structure and Function in a Novel Model of Experimental Rhegmatogenous Retinal Detachment. Invest Ophthalmol Vis Sci. 2023; 64 (5): 3. doi:10.1167/iovs.64.5.3
21. Rice G I, Meyzer C, Bouazza N, et al. Reverse-Transcriptase Inhibitors in the Aicardi-Goutières Syndrome. N Engl J Med. 2018; 379 (23): 2275-2277. doi:10.1056/NEJMc1810983
22. Thorlund K, Dron L, Park J J H, Mills E J. Synthetic and External Controls in Clinical Trials-A Primer for Researchers. Clin Epidemiol. 2020; 12:457-467. doi:10.2147/CLEP.S242097
23. Dugel P U, Hillenkamp J, Sivaprasad S, et al. Baseline visual acuity strongly predicts visual acuity gain in patients with diabetic macular edema following anti-vascular endothelial growth factor treatment across trials. Clin Ophthalmol. 2016; 10:1103-1110. doi:10.2147/OPTH.S100764
24. Bressler N M, Odia I, Maguire M, et al. Association Between Change in Visual Acuity and Change in Central Subfield Thickness During Treatment of Diabetic Macular Edema in Participants Randomized to Aflibercept, Bevacizumab, or Ranibizumab: A Post Hoc Analysis of the Protocol T Randomized Clinical Trial. JAMA Ophthalmol. 2019; 137 (9): 977-985. doi:10.1001/jamaophthalmol.2019.1963
25. Wang P, Hu Z, Hou M, Norman P A, Chin E K, Almeida D R. Relationship Between Macular Thickness and Visual Acuity in the Treatment of Diabetic Macular Edema With Anti-VEGF Therapy: Systematic Review. J Vitreoretin Dis. 2023; 7 (1): 57-64. doi:10.1177/24741264221138722
26. Nair P, Aiello L P, Gardner T W, Jampol L M, Ferris F L. Report From the NEI/FDA Diabetic Retinopathy Clinical Trial Design and Endpoints Workshop. Invest Ophthalmol Vis Sci. 2016; 57 (13): 5127-5142. doi:10.1167/iovs.16-20356
27. Lewis W, Dalakas M C. Mitochondrial toxicity of antiviral drugs. Nat Med. 1995; 1 (5): 417-422. doi:10.1038/nm0595-417
28. Narendran S, Pereira F, Yerramothu P, et al. Nucleoside reverse transcriptase inhibitors and Kamuvudines inhibit amyloid-β induced retinal pigmented epithelium degeneration. Signal Transduct Target Ther. 2021; 6 (1): 149. doi:10.1038/s41392-021-00537-z

One of ordinary skill in the art will recognize that additional embodiments or implementations are possible without departing from the teachings of the present disclosure or the scope of the claims which follow. This detailed description, and particularly the specific details of the exemplary embodiments and implementations disclosed herein, is given primarily for clarity of understanding, and no unnecessary limitations are to be understood therefrom, for modifications will become obvious to those skilled in the art upon reading this disclosure and may be made without departing from the scope of the claimed invention.

All publications, patents, and patent applications, Genbank sequences, websites and other published materials referred to throughout the disclosure herein are herein incorporated by reference to the same extent as if each individual publication, patent, or patent application, Genbank sequences, websites and other published materials was specifically and individually indicated to be incorporated by reference. In the event that the definition of a term incorporated by reference conflicts with a term defined herein, this specification shall control.

1. A method to prevent or treat a retinal vascular disease comprising administering one or more inflammasome inhibitors to a subject in need thereof.

2. The method of claim 1, further comprising administering one or more vascular endothelial growth factor (VEGF) inhibitors.

3. The method of claim 1, wherein the one or more inflammasome inhibitors comprises a nucleotide reverse transcriptase inhibitor (NRTI) or an NRTI derivative.

4. The method of claim 3, wherein the NRTI derivative comprises an alkylated NRTI.

5. The method of claim 3, wherein the NRTI or alkylated NRTI comprises kamuvudine-9 (K-9), lamivudine (3TC), azidothymidine (AZT), kamuvudine-8 (K-8; 2-ethyl-AZT) or a combination thereof.

6. The method of claim 3, wherein the administered dose of the NRTI or NRTI derivative is equal to or less than 150 mg once or twice daily.

7. The method of claim 2, wherein the one or more VEGF inhibitors comprises aflibercept, bevacizumab, ranibizumab, onbercept, ziv-aflibercept, Razumab, Bvooviz, Cimerli, Xlucane, R-TPR-024, SIP-0133, UBTO10, CKD-701, SB15, MYL1701, ABP-938, FVB203, SOK583419, CT-P42, ALT-L9, OT-702 or Bevacizumab-vikg.

8. The method of claim 1, wherein the one or more inflammasome inhibitors and/or the one or more VEGF inhibitors are administered orally or injected.

9. The method of claim 8, wherein the one or more inflammasome inhibitors and/or the one or more VEGF inhibitors are injected in the eye of the subject.

10. The method of claim 1, wherein one or more inflammasome inhibitors comprises a compound of structural Formula (I) 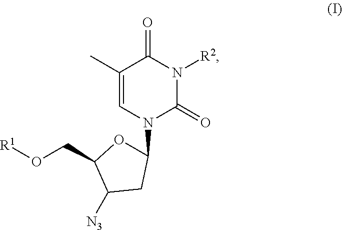
wherein:
R¹ is C₁₋₄ alkyl; and
R² is H or C₁₋₄ alkyl, provided that when R² is H, R¹ is not CH₃ or a salt thereof.

11. The method of claim 10, wherein R² is CH₃ or CH₂CH₃.

12. The method of claim 10, wherein R¹ is n-C₄H₉.

13. The method of claim 1, wherein the one or more inflammasome inhibitors comprises one or more of 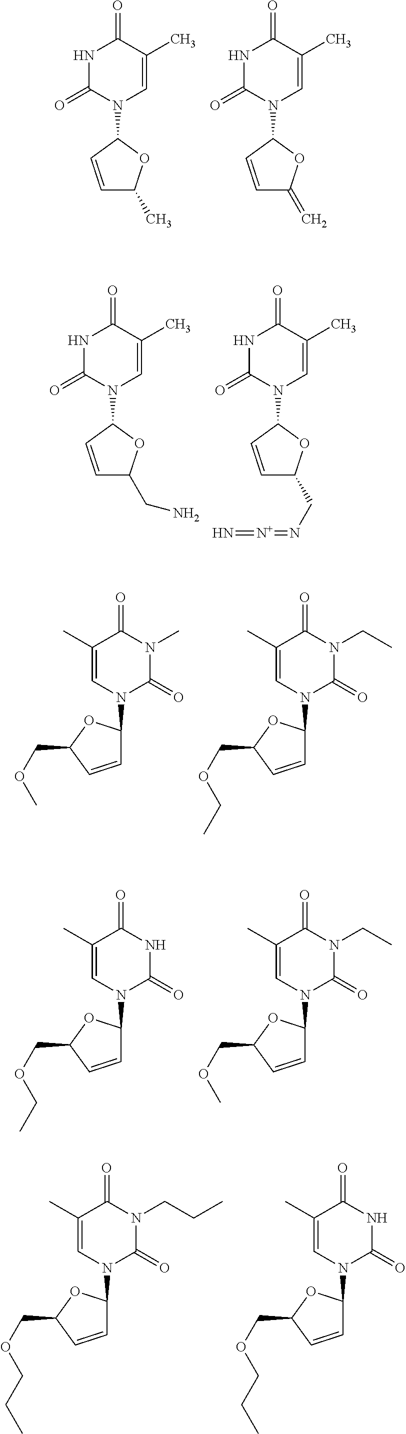 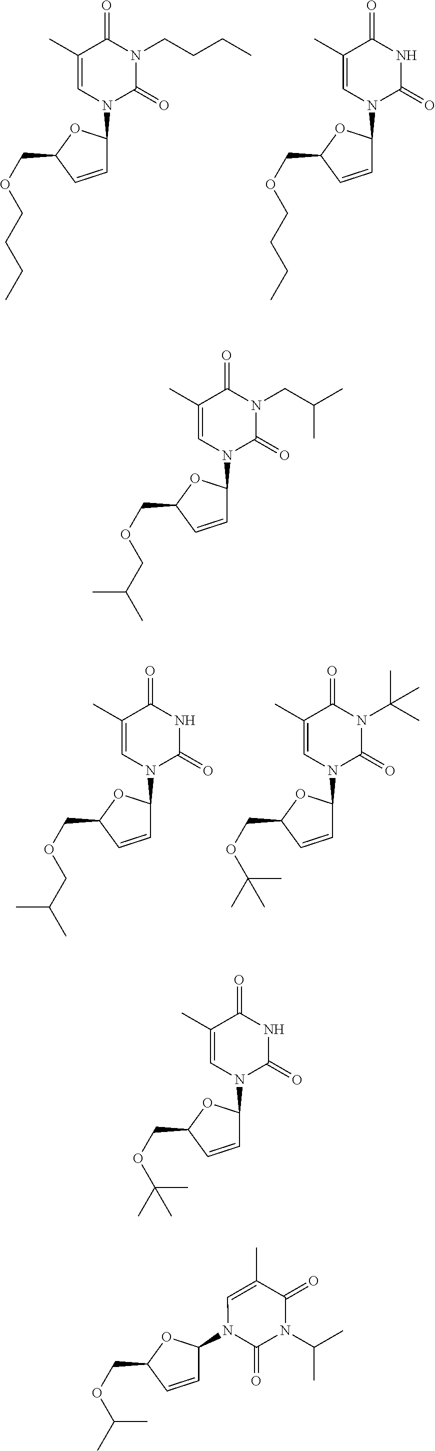 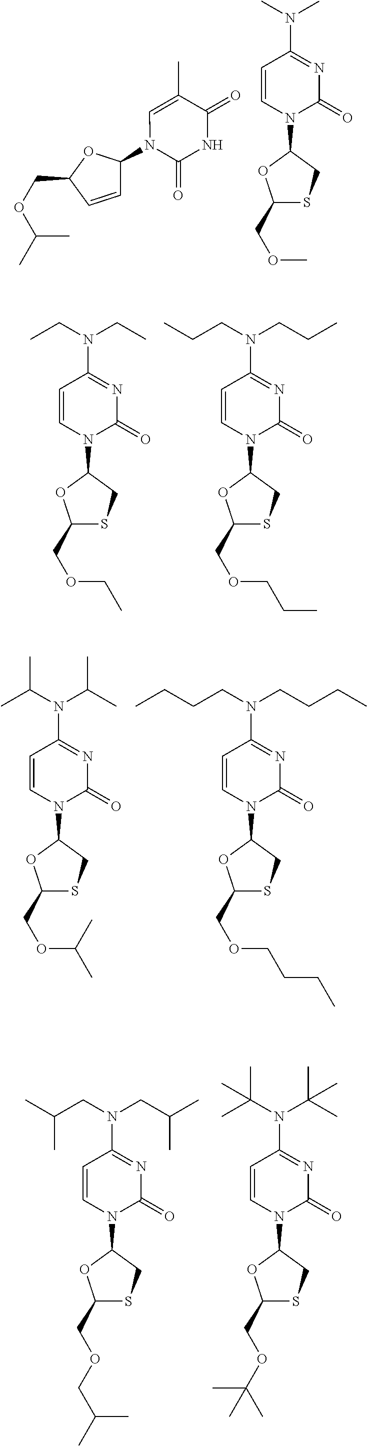 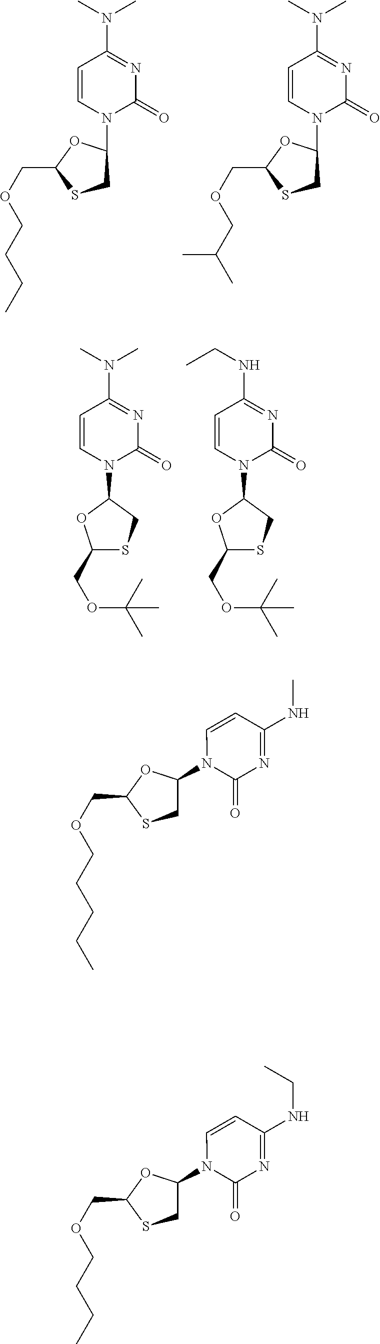 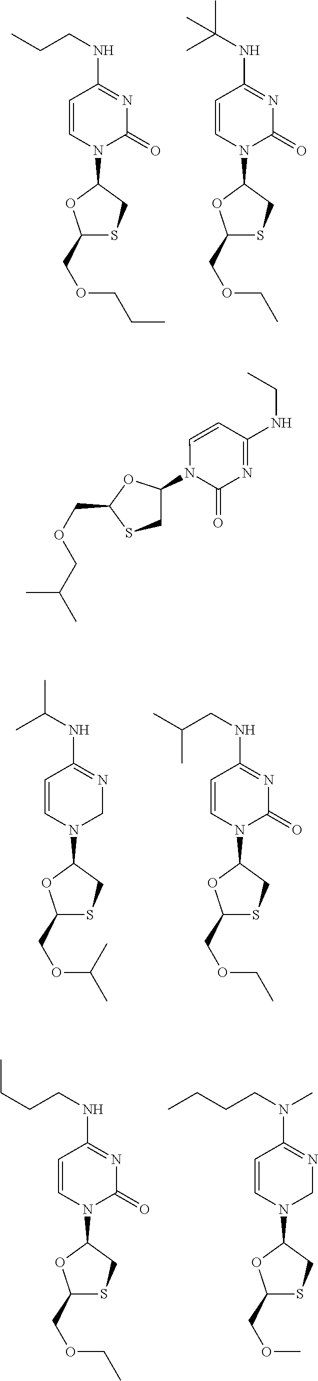 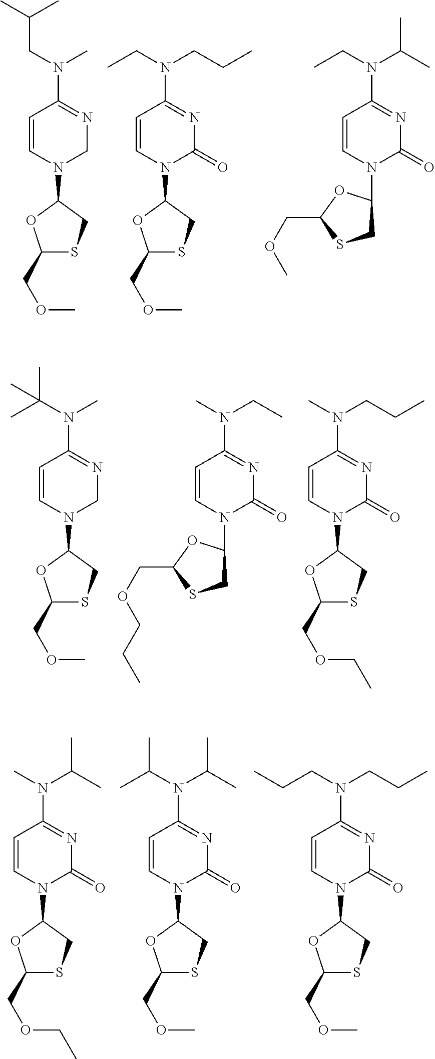 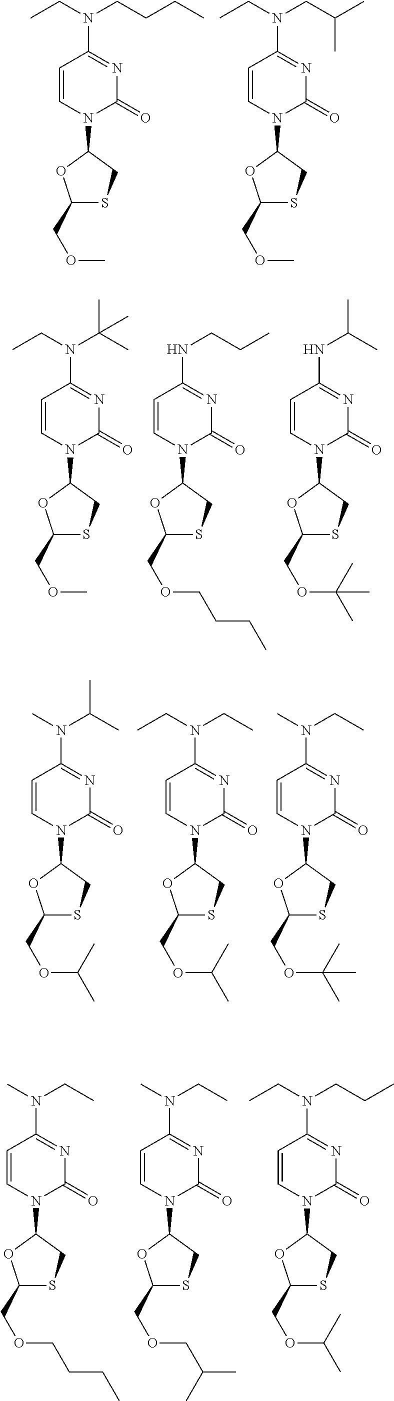 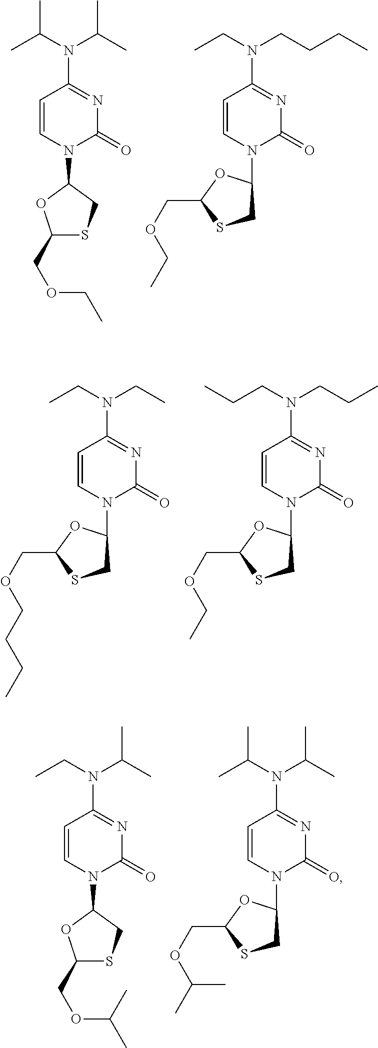 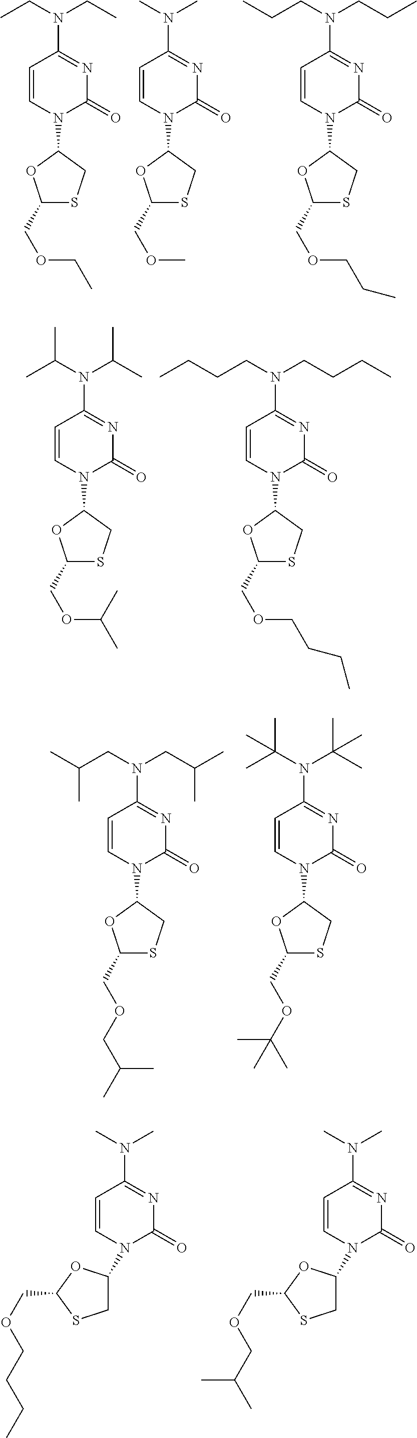 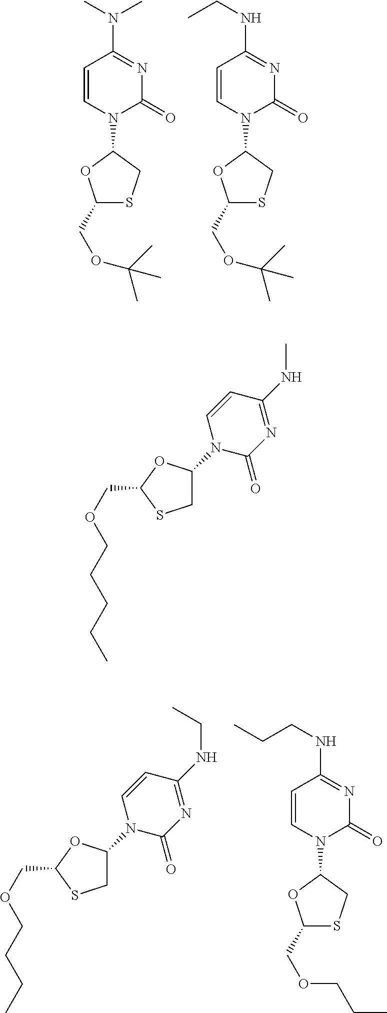 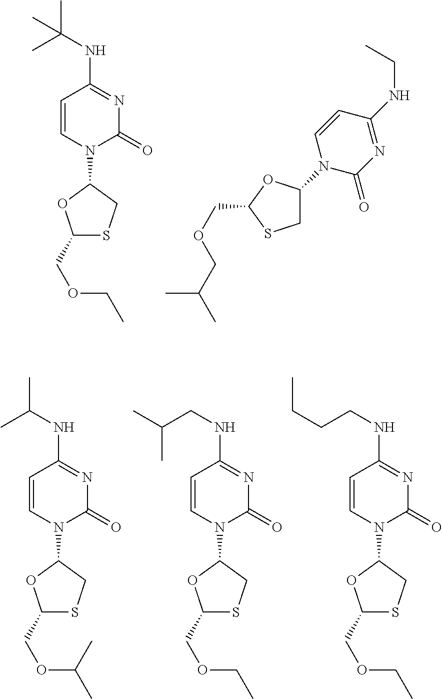 [SMILES: CCOC[C@@H]1O[C@H](n2ccc(NC(C)(C)C)nc2=O)CS1 ; CCNc1ccn([C@@H]2CS[C@H](COCC(C)C)O2)c(=O)n1 ; CC(C)Nc1ccn([C@@H]2CS[C@H](COC(C)C)O2)c(=O)n1 ; CCOC[C@@H]1O[C@H](n2ccc(NCC(C)C)nc2=O)CS1 ; CCCCNc1ccn([C@@H]2CS[C@H](COCC)O2)c(=O)n1] 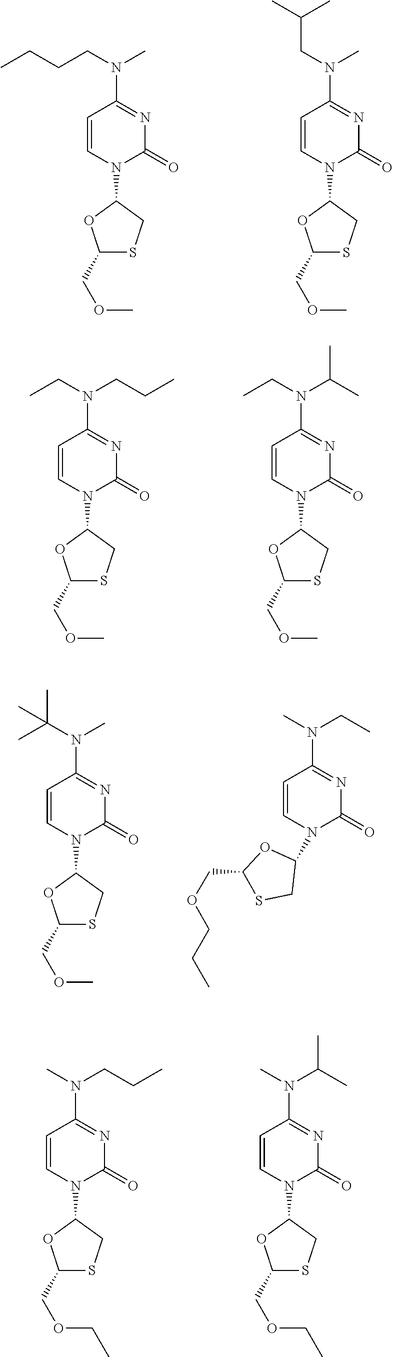 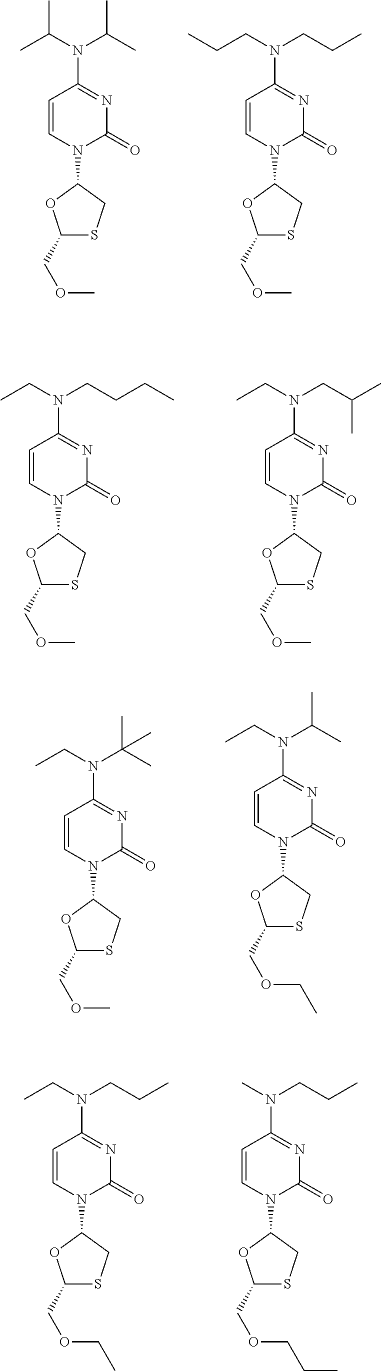 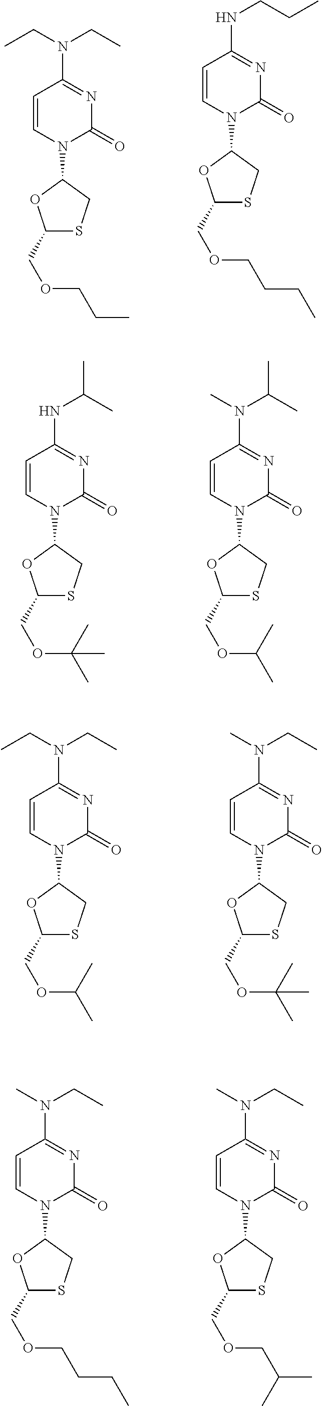 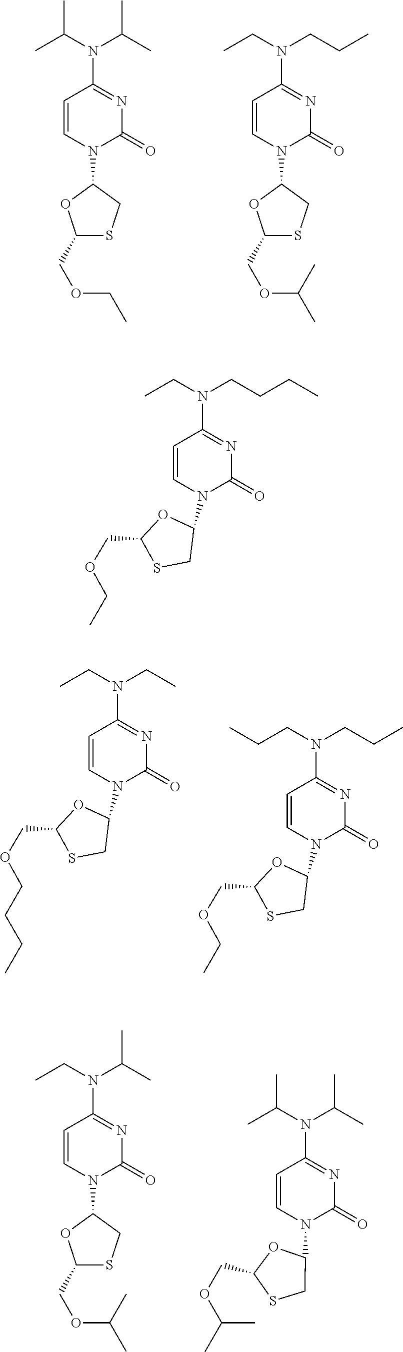 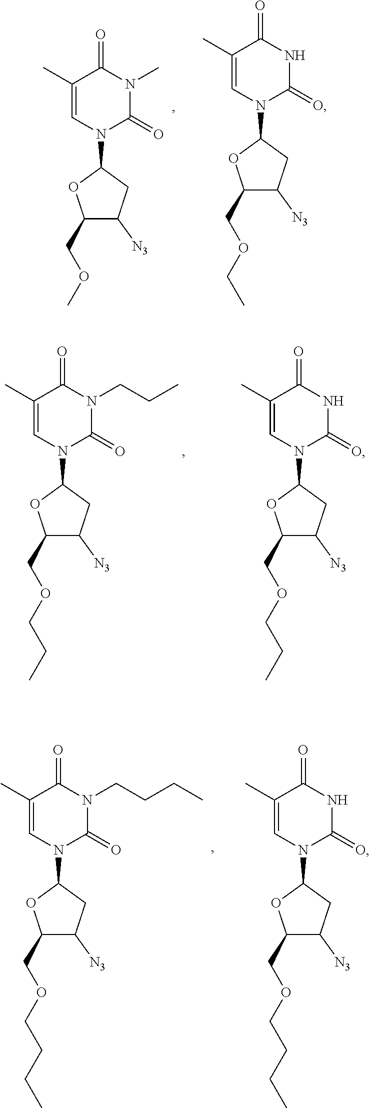 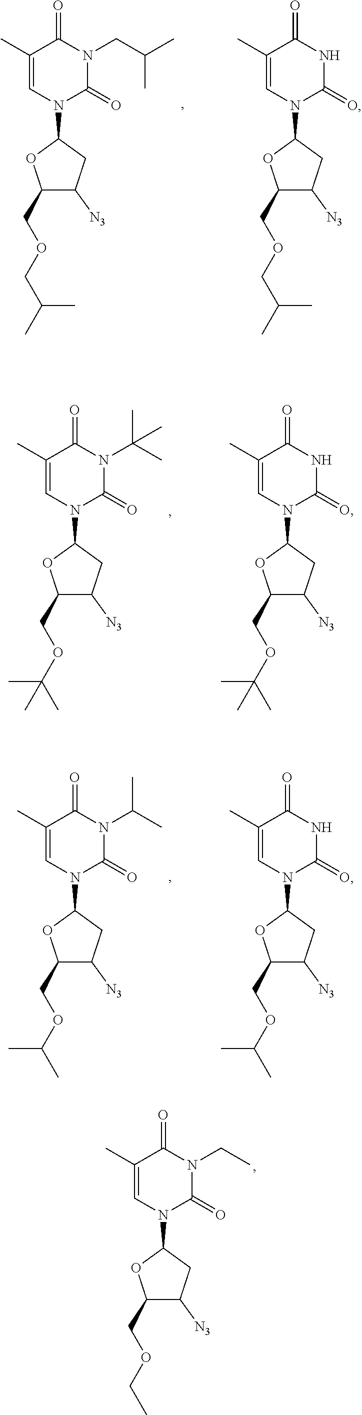
an enantiomer or a pharmaceutically acceptable salt thereof.

14. The method of claim 1, wherein the vascular retinal disease is diabetic macular edema (DME), branch or central retinal vein occlusion (RVO), neovascular age-related macular degeneration (“wet” AMD), diabetic retinopathy, macular edema due to retinitis pigmentosa, Coats disease, sickle cell disease, polypoidal choroidal vasculopathy or macular neovascularization, either as NRTI or NRTI derivative monotherapy or in combination with anti-VEGF agents.

15. The method of claim 1, wherein the treatment results in an improvement in visual acuity.

16. The method of claim 3, wherein the NRTI or NRTI derivative is administered by intravitreous injection to said subject.

17. The method of claim 2, wherein the one or more VEGF inhibitors are administered by intravitreous injection to said subject either before, after or at the same time as administration of the NRTI or NRTI derivative.
